(19) United States
(12) Patent Application Publication
(10) Pub. No.: US 2026/0191898 A1
(43) Pub. Date: Jul. 9, 2026

(54) COMPOSITIONS AND METHODS FOR TREATING DISEASES RESPONSIVE TO AKG-VITAMIN B COADMINISTRATION

(71) Applicants: Robb D. FINNEMORE, Fernandina Beach, FL (US); Thomas D. WELDON, Novato, CA (US); Francis R. PALMER, III, Beverly Hills, CA (US); PONCE DE LEON HEALTH DESIGNATED ACTIVITY COMPANY, Drogheda, County Louth (IE)

(72) Inventors: Robb D. Finnemore, Fernandina Beach, FL (US); Thomas D. Weldon, Novato, CA (US); Francis R. PALMER, III, Santa Monica, CA (US)

(21) Appl. No.: 19/132,079

(22) PCT Filed: Dec. 5, 2023
(86) PCT No.: PCT/US2023/082444

§ 371 Date: May 22, 2025

Related U.S. Application Data
(60) Provisional application No. 63/430,519, filed on Dec. 6, 2022.
(60) Provisional application No. 63/464,072, filed on May 4, 2023.

(51) Int. Cl. A61K 31/714; A61K 31/194; A61K 31/4415; A61K 31/525; A61P 43/00

(52) U.S. Cl. CPC A61K 31/714; A61K 31/194; A61K 31/4415; A61K 31/525; A61P 43/00

(57) ABSTRACT

Compositions and methods for treating diseases that are responsive to the coadministration of AKG and certain B vitamins are described herein. In addition, compositions and methods for treating host animals that may not respond to AKG monotherapy, including treating host animals having inadequate or dysfunctional expression of enzymes involved in vitamin B metabolism and homeostasis, such as methylenetetrahydrofolate reductase (MTHFR), are described herein.

### TECHNICAL FIELD

The invention described herein pertains to compositions and methods for treating diseases that are responsive to the coadministration of AKG and certain B vitamins. In addition, the invention described herein pertains to compositions and methods for treating host animals that may not respond to AKG monotherapy. In addition, the invention described herein pertains to compositions and methods for treating host animals having inadequate or dysfunctional expression of enzymes involved in vitamin B metabolism and homeostasis, such as methylenetetrahydrofolate reductase (MTHFR).

### BACKGROUND AND SUMMARY OF THE INVENTION

Calcium α-ketoglutarate (Ca-AKG) has been reported to be efficacious in treating age-related illness. However, it has been observed herein that patients that have certain B vitamin abnormalities, mutations in certain enzymes involved in the regulation of B vitamins, and/or mutations in certain enzymes involved in the regulation of B vitamins that lead to B vitamin abnormalities show an insensitivity to Ca-AKG treatment. B vitamin abnormalities include conditions where the subject has an insufficient amount of one or more biologically available B vitamins, including one or more B vitamin deficiencies. It has been discovered that herein that such patients having one or more B vitamin deficiencies show an improved response to Ca-AKG treatment when co-administered with one or more B vitamins selected from vitamins B2, B6, B9, and B12, and combinations thereof.

In one illustrative embodiment of the invention described herein, methods for treating age-related illness are described. In another illustrative embodiment described herein, methods for increasing longevity are described. The methods include co-administering a therapeutically effective amount of an α-ketoglutarate (AKG) and one or more B vitamins. In another illustrative embodiment, uses of compositions comprising a therapeutically effective amount of an α-ketoglutarate (AKG) and one or more B vitamins are described herein for the manufacture of a medicament for treating age-related illness. In another embodiment, compositions comprising a therapeutically effective amount of an α-ketoglutarate (AKG) and one or more B vitamins are described herein for use in the treatment of age-related illness. In another embodiment, unit doses comprising a therapeutically effective amount of an α-ketoglutarate (AKG) and one or more B vitamins are described herein for use in the treatment of age-related illness. In another embodiment, kits comprising a therapeutically effective amount of an α-ketoglutarate (AKG), one or more B vitamins, and instructions are described herein for use in the treatment of age-related illness.

In another embodiment, methods for treating age-related illness and/or increasing longevity in a subject having a vitamin B9 metabolism mutation are described, where the methods include coadministering a therapeutically effective amount of an α-ketoglutarate (AKG) and one or more folates to the subject. In another embodiment, uses of compositions comprising a therapeutically effective amount of an α-ketoglutarate (AKG) and one or more B vitamins are described herein for the manufacture of a medicament for treating age-related illness in a subject having a vitamin B9 metabolism mutation are described. In another embodiment, compositions comprising a therapeutically effective amount of an α-ketoglutarate (AKG) and one or more B vitamins are described herein for use in the treatment of age-related illness in a subject having a vitamin B9 metabolism. In another embodiment, unit doses comprising a therapeutically effective amount of an α-ketoglutarate (AKG) and one or more B vitamins are described herein for use in the treatment of age-related illness in a subject having a vitamin B9 metabolism. In another embodiment, kits comprising a therapeutically effective amount of an α-ketoglutarate (AKG), one or more B vitamins, and instructions are described herein for use in the treatment of age-related illness in a subject having a vitamin B9 metabolism. In another embodiment, the vitamin B9 metabolism mutation includes mutation of methylenetetrahydrofolate reductase (MTHFR). In another embodiment, the one or more B vitamins include vitamin B9. In another embodiment, the one or more B vitamins include vitamin B2.

In another embodiment, methods for treating age-related illness and/or increasing longevity in a subject having a vitamin B6 metabolism mutation are described, where the methods include coadministering a therapeutically effective amount of an α-ketoglutarate (AKG) and vitamin B6 to the subject. In another embodiment, uses of compositions comprising a therapeutically effective amount of an α-ketoglutarate (AKG) and one or more B vitamins are described herein for the manufacture of a medicament for treating age-related illness in a subject having a vitamin B6 metabolism mutation are described. In another embodiment, compositions comprising a therapeutically effective amount of an α-ketoglutarate (AKG) and one or more B vitamins are described herein for use in the treatment of age-related illness in a subject having a vitamin B6 metabolism. In another embodiment, unit doses comprising a therapeutically effective amount of an α-ketoglutarate (AKG) and one or more B vitamins are described herein for use in the treatment of age-related illness in a subject having a vitamin B6 metabolism. In another embodiment, kits comprising a therapeutically effective amount of an α-ketoglutarate (AKG), one or more B vitamins, and instructions are described herein for use in the treatment of age-related illness in a subject having a vitamin B6 metabolism. In another embodiment, the vitamin B6 metabolism mutation includes mutation of Alkaline Phosphatase (ALPL). In another embodiment, the one or more B vitamins include vitamin B6 or vitamin B2, or a combination of vitamins B6 and B2.

In another embodiment, methods for treating age-related illness and/or increasing longevity in a subject exhibiting reduced vitamin B12 absorption are described, where the methods include coadministering a therapeutically effective amount of an α-ketoglutarate (AKG) and vitamin B12 to the subject. In another embodiment, uses of compositions comprising a therapeutically effective amount of an α-ketoglutarate (AKG) and one or more B vitamins are described herein for the manufacture of a medicament for treating age-related illness in a subject exhibiting reduced vitamin B12 absorption are described. In another embodiment, compositions comprising a therapeutically effective amount of an α-ketoglutarate (AKG) and one or more B vitamins are described herein for use in the treatment of age-related illness in a subject exhibiting reduced vitamin B12 absorption. In another embodiment, unit doses comprising a therapeutically effective amount of an α-ketoglutarate (AKG) and one or more B vitamins are described herein for use in the treatment of age-related illness in a subject exhibiting reduced vitamin B12 absorption. In another embodiment, kits comprising a therapeutically effective amount of an α-ketoglutarate (AKG), one or more B vitamins, and instructions are described herein for use in the treatment of age-related illness in a subject exhibiting reduced vitamin B12 absorption. In another embodiment, the one or more B vitamins include vitamin B12. In another embodiment, the one or more B vitamins include vitamin B2. It is understood herein that subjects exhibiting reduced vitamin B12 absorption include geriatric patients, such as subjects 65 year old and older.

In another embodiment, methods for treating age-related illness and/or increasing longevity in a subject having a vitamin B12 metabolism mutation are described, where the methods include coadministering a therapeutically effective amount of an α-ketoglutarate (AKG) and vitamin B12 to the subject. In another embodiment, uses of compositions comprising a therapeutically effective amount of an α-ketoglutarate (AKG) and one or more B vitamins are described herein for the manufacture of a medicament for treating age-related illness in a subject having a vitamin B12 metabolism mutation are described. In another embodiment, compositions comprising a therapeutically effective amount of an α-ketoglutarate (AKG) and one or more B vitamins are described herein for use in the treatment of age-related illness in a subject having a vitamin B12 metabolism. In another embodiment, unit doses comprising a therapeutically effective amount of an α-ketoglutarate (AKG) and one or more B vitamins are described herein for use in the treatment of age-related illness in a subject having a vitamin B12 metabolism. In another embodiment, kits comprising a therapeutically effective amount of an α-ketoglutarate (AKG), one or more B vitamins, and instructions are described herein for use in the treatment of age-related illness in a subject having a vitamin B12 metabolism. In another embodiment, the vitamin B12 metabolism mutation includes mutation of Fucosyltransferase (FUT2). In another embodiment, the one or more B vitamins include vitamin B12. In another embodiment, the one or more B vitamins include vitamin B2.

In another embodiment, the methods, uses, compositions, unit dose, and kits described herein delay and/or reverse an age-related illness, and/or increasing longevity. Illustratively, the age-related illness comprises frailty, hair loss, loss of hair pigmentation, osteoporosis, obesity, type-2 diabetes, macular degeneration, or an autoimmune disease, sarcopenia, impaired cognition, decreased stem cell production, increased inflammation, age-related epigenetic changes, and/or epigenetic changes in DNA methylation profile. Illustratively, improvements or decreases in frailty include, but are not limited to, improved locomotion, gait, and/or balance, improved and/or increased exercise efficiently, and/or improved and/or increased exercise endurance. Additional details regarding age-related illness are described in WO 2018/200736 and WO 2020/252005, the entire disclosures of which are incorporated herein by reference.

In another embodiment, pharmaceutical compositions containing one or more of the compounds are also described herein. In another embodiment, the compositions include a therapeutically effective amount of the one or more compounds for treating a host animal with a cancer. It is to be understood that the compositions may include other components and/or ingredients, including, but not limited to, other therapeutically active compounds, and/or one or more carriers, vehicles, diluents, adjuvants, excipients, and the like, and combinations thereof.

In another embodiment, unit doses of the compounds and pharmaceutical compositions containing one or more of the compounds are also described herein. The unit doses include a therapeutically effective amount of the one or more compounds for treating a host animal with an age-related illness. The unit doses are in single or divided form, and may correspond to a daily dosage amount, or adjusted to a periodic amount that is shorter, including for multiple daily doses, or longer, including weekly or monthly doses. It is to be understood that the compositions may include other components and/or ingredients, including, but not limited to, other therapeutically active compounds, and/or one or more carriers, vehicles, diluents, adjuvants, excipients, and the like, and combinations thereof.

### BRIEF DESCRIPTION OF THE DRAWINGS

FIG. 1 shows the decrease in true biological age resulting from treatment with co-administration of Ca-AKG and vitamin B complex.

### DETAILED DESCRIPTION

Several illustrative embodiments of the invention are described by the following enumerated clauses:

Use of Ca-AKG and one or more B vitamins in the manufacture of a medicament for treating age-related illness and/or increasing longevity in a host animal, subject, or patient.

A pharmaceutical composition comprising Ca-AKG and one or more B vitamins for use in treating age-related illness and/or increasing longevity in a host animal, subject, or patient.

A method for treating age-related illness and/or increasing longevity in a host animal, subject, or patient, the method comprising administering a therapeutically effective amount of a combination of Ca-AKG and one or more B vitamins to the host animal, subject, or patient.

A unit dose or unit dosage form in single or divided form, the unit dose or unit dosage comprising a therapeutically effective amount of Ca-AKG and one or more B vitamins, and adapted for or configured for use in treating age-related illness and/or increasing longevity in a host animal, subject, or patient.

A kit comprising a therapeutically effective amount of Ca-AKG, one or more B vitamins, and instructions for treating age-related illness and/or increasing longevity in a host animal, subject, or patient.

The use, composition, method, unit dose, or kit according to any one of the preceding clauses wherein the B vitamin is vitamin B2.

The use, composition, method, unit dose, or kit according to any one of the preceding clauses wherein the B vitamin is vitamin B6.

The use, composition, method, unit dose, or kit according to any one of the preceding clauses wherein the B vitamin is vitamin B9.

The use, composition, method, unit dose, or kit according to any one of the preceding clauses wherein the B vitamin is vitamin B12.

The use, composition, method, unit dose, or kit according to any one of the preceding clauses wherein the age-related illness is frailty.

The use, composition, method, unit dose, or kit according to any one of the preceding clauses wherein the host animal, subject, or patient is a human.

The use, composition, method, unit dose, or kit according to any one of the preceding clauses wherein the host animal, subject, or patient is a human male.

The use, composition, method, unit dose, or kit according to any one of the preceding clauses wherein the host animal, subject, or patient is a human female.

The use, composition, method, unit dose, or kit according to any one of the preceding clauses wherein the host animal, subject, or patient exhibits a deficiency in blood levels of vitamin B6.

The use, composition, method, unit dose, or kit according to any one of the preceding clauses wherein the host animal, subject, or patient exhibits a deficiency in blood levels of vitamin B12.

The use, composition, method, unit dose, or kit according to any one of the preceding clauses wherein the host animal, subject, or patient has single polynucleotide polymorphism in MTHFR.

The use, composition, method, unit dose, or kit according to any one of the preceding clauses wherein the single polynucleotide polymorphism in MTHFR is A/A, homozygous positive.

The use, composition, method, unit dose, or kit according to any one of the preceding clauses wherein the single polynucleotide polymorphism in MTHFR is A/G, heterozygous positive.

The use, composition, method, unit dose, or kit according to any one of the preceding clauses wherein the single polynucleotide polymorphism in MTHFR is C667T.

The use, composition, method, unit dose, or kit according to any one of the preceding clauses wherein the single polynucleotide polymorphism in MTHFR is A1298C.

The use, composition, method, unit dose, or kit according to any one of the preceding clauses wherein the host animal, subject, or patient has single polynucleotide polymorphism in ALPL.

The use, composition, method, unit dose, or kit according to any one of the preceding clauses wherein the single polynucleotide polymorphism in ALPL is C/C, homozygous positive.

The use, composition, method, unit dose, or kit according to any one of the preceding clauses wherein the single polynucleotide polymorphism in ALPL is C/T, heterozygous positive.

The use, composition, method, unit dose, or kit according to any one of the preceding clauses wherein the host animal, subject, or patient has single polynucleotide polymorphism in FUT2.

The use, composition, method, unit dose, or kit according to any one of the preceding clauses wherein the single polynucleotide polymorphism in ALPL is G/G, homozygous positive.

The use, composition, method, unit dose, or kit according to any one of the preceding clauses wherein the single polynucleotide polymorphism in ALPL is A/G, heterozygous positive.

The use, composition, method, unit dose, or kit according to any one of the preceding clauses wherein the therapeutically effective amount is sufficient to decrease the biological age of the host animal, subject, or patient.

The use, composition, method, unit dose, or kit according to any one of the preceding clauses wherein the therapeutically effective amount is sufficient to improve the DNA methylation profile of the host animal, subject, or patient.

The use, composition, method, unit dose, or kit according to any one of the preceding clauses wherein the decrease in the biological age of the host animal, subject, or patient is determined by the DNA methylation profile of the host animal, subject, or patient.

The use, composition, method, unit dose, or kit according to any one of the preceding clauses further comprising a vitamin D, such as vitamin D3.

The use, composition, method, unit dose, or kit according to any one of the preceding clauses further comprising a vitamin A, such as retinyl palmitate.

A method for treating an age-related disease or illness in a host animal, the method comprising administering to the host animal a predetermined amount of an alpha-ketoglutaratic acid or salt thereof, and a predetermined amount of a B vitamin or salt thereof, where the predetermined amounts together are therapeutically effective in treating the age-related disease.

A composition comprising an alpha-ketoglutaratic acid or salt thereof, and a B vitamin or salt thereof, and optionally including one or more carriers, diluents, excipients, and the like, and combinations thereof.

A kit adapted for treating an age-related disease in a host animal, the kit comprising a dose of an alpha-ketoglutaratic acid or salt thereof, a dose of a B vitamin or salt thereof, and instructions for administering the dose of the alpha-ketoglutaratic acid or salt thereof and the dose of the B vitamin or salt thereof to the host animal for treating the age-related disease.

A pharmaceutical composition comprising a compound of any of the clauses recited herein, and optionally comprising one or more carriers, diluents, excipients, and the like, and combinations thereof.

A pharmaceutical composition comprising a compound of any of the clauses recited herein, and optionally comprising one or more carriers, diluents, excipients, and the like, and combinations thereof for use in treating an age-related disease in a host animal.

A unit dose comprising a compound of any of the clauses recited herein, or a pharmaceutical composition thereof and optionally comprising one or more carriers, diluents, excipients, and the like, and combinations thereof, where the unit dose is in single or divided form, and includes a therapeutically effective amount of the compound for treating a host animal with an age-related disease.

A method for treating an age-related disease in a host animal, the method comprising administering a therapeutically effective amount of a compound of any of the clauses recited herein, or a pharmaceutical composition thereof and optionally comprising one or more carriers, diluents, excipients, and the like, and combinations thereof, or a unit dose comprising any of the foregoing, in single or divided form, to the host animal.

Use of a compound of any of the clauses recited herein, or a pharmaceutical composition thereof and optionally comprising one or more carriers, diluents, excipients, and the like, and combinations thereof, in the manufacture of a medicament, including a unit dose, in single or divided form, for treating a host animal with an age-related disease.

A kit comprising the compound, composition, or unit dose, of any one of the preceding clauses; an optional carrier; an optional container for preparing a composition for administration comprising the compound or composition or unit dose and the optional carrier; and instructions for administering the compound or composition or unit dose or composition for administration to a host animal having an age-related disease.

In each of the foregoing and each of the following embodiments, unless otherwise indicated, it is also to be understood that the transitional phrase “consisting essentially of” means that the scope of the corresponding composition, unit dose, method or use is understood to encompass the specified compounds or recited steps, and those that do not materially affect the basic and novel characteristics of the invention described herein. For example, a method described herein that consists essentially of a single compound, or genus of compounds, is understood to represent a monotherapy for the recited disease. Though the monotherapy may include co-administration of one or more carriers, vehicles, diluents, adjuvants, excipients, and the like, and combinations thereof, and/or include co-administration of one or more additional active pharmaceutical ingredients, those latter additional active pharmaceutical ingredients are to be understood to be for treating diseases and/or symptoms distinct from treating the underlying conditions described herein, such as the age-related illness itself.

In another embodiment, the therapeutically effective amount of Ca-AKG is at least 5 mg/kg, at least 10 mg/kg, at least 15 mg/kg, at least 16 mg/kg, at least 16.1 mg/kg, at least 16.2 mg/kg, at least 16.2 mg/kg, at least 16.4 mg/kg, at least 16.5 mg/kg, at least 16.6 mg/kg, at least 16.7 mg/kg, at least 16.8 mg/kg, at least 16.9 mg/kg, at least 17 mg/kg, at least 18 mg/kg, at least 19 mg/kg, at least 20 mg/kg, at least 21 mg/kg, at least 22 mg/kg, at least 23 mg/kg, at least 24 mg/kg, at least 25 mg/kg, at least 28 mg/kg, at least 30 mg/kg, or at least 33 mg/kg.

In another embodiment, the amount of calcium alpha-ketoglutarate is from 50 mg to 5000 mg. In another embodiment, the amount of calcium alpha-ketoglutarate is from 100 mg to 2000 mg. In another embodiment, the therapeutically effective amount of Ca-AKG is at least 350 mg and no greater than 2000 mg. In another embodiment, the therapeutically effective amount of Ca-AKG is about 350 mg to about 2000 mg. In another embodiment, the therapeutically effective amount of Ca-AKG is about 400 mg to about 2000 mg. In another embodiment, the therapeutically effective amount of Ca-AKG is about 450 mg to about 2000 mg. In another embodiment, the therapeutically effective amount of Ca-AKG is about 500 mg to about 2000 mg. In another embodiment, the therapeutically effective amount of Ca-AKG is about 550 mg to about 2000 mg. In another embodiment, the therapeutically effective amount of Ca-AKG is about 600 mg to about 2000 mg. In another embodiment, the therapeutically effective amount of Ca-AKG is about 650 mg to about 2000 mg. In another embodiment, the therapeutically effective amount of Ca-AKG is about 700 mg to about 2000 mg. In another embodiment, the therapeutically effective amount of Ca-AKG is about 800 mg to about 2000 mg. In another embodiment, the therapeutically effective amount of Ca-AKG is about 900 mg to about 2000 mg. In another embodiment, the therapeutically effective amount of Ca-AKG is about 1000 mg to about 2000 mg. In another embodiment, the therapeutically effective amount of Ca-AKG is about 1100 mg to about 2000 mg. In another embodiment, the therapeutically effective amount of Ca-AKG is about 1200 mg to about 2000 mg. In another embodiment, the therapeutically effective amount of Ca-AKG is about 1300 mg to about 2000 mg. In another embodiment, the therapeutically effective amount of Ca-AKG is about 1400 mg to about 2000 mg. In another embodiment, the therapeutically effective amount of Ca-AKG is about 1500 mg to about 2000 mg. In another embodiment, the therapeutically effective amount of Ca-AKG is about 1600 mg to about 2000 mg. In another embodiment, the therapeutically effective amount of Ca-AKG is about 1700 mg to about 2000 mg. In another embodiment, the therapeutically effective amount of Ca-AKG is about 1800 mg to about 2000 mg. In another embodiment, the therapeutically effective amount of Ca-AKG is about 1900 mg to about 2000 mg. In another embodiment, the therapeutically effective amount of Ca-AKG is about 350 mg to about 1900 mg. In another embodiment, the therapeutically effective amount of Ca-AKG is about 350 mg to about 1800 mg. In another embodiment, the therapeutically effective amount of Ca-AKG is 350 mg to about 1700 mg. In another embodiment, the therapeutically effective amount of Ca-AKG is about 350 mg to about 1600 mg. In another embodiment, the therapeutically effective amount of Ca-AKG is about 350 mg to about 1500 mg. In another embodiment, the therapeutically effective amount of Ca-AKG is about 350 mg to about 1400 mg. In another embodiment, the therapeutically effective amount of Ca-AKG is about 350 mg to about 1300 mg. In another embodiment, the therapeutically effective amount of Ca-AKG is about 350 mg to about 1200 mg. In another embodiment, the therapeutically effective amount of Ca-AKG is about 350 mg to about 1100 mg. In another embodiment, the therapeutically effective amount of Ca-AKG is about 350 mg to about 1000 mg. In another embodiment, the therapeutically effective amount of Ca-AKG is about 350 mg to about 900 mg. In another embodiment, the therapeutically effective amount of Ca-AKG is about 350 mg to about 800 mg. In another embodiment, the therapeutically effective amount of Ca-AKG is about 350 mg to about 700 mg. In another embodiment, the therapeutically effective amount of Ca-AKG is bout 350 mg to about 650 mg. In another embodiment, the therapeutically effective amount of Ca-AKG is about 350 mg to about 600 mg. In another embodiment, the therapeutically effective amount of Ca-AKG is about 350 mg to about 550 mg. In another embodiment, the therapeutically effective amount of Ca-AKG is about 350 mg to about 500 mg. In another embodiment, the therapeutically effective amount of Ca-AKG is about 350 mg to about 450 mg. In another embodiment, the therapeutically effective amount of Ca-AKG is about 350 mg to about 400 mg. In another embodiment, the therapeutically effective amount of Ca-AKG is at least about 350 mg. In another embodiment, the therapeutically effective amount of Ca-AKG is at least about 400 mg. In another embodiment, the therapeutically effective amount of Ca-AKG is at least about 450 mg. In another embodiment, the therapeutically effective amount of Ca-AKG is at least about 500 mg. In another embodiment, the therapeutically effective amount of Ca-AKG is at least about 550 mg. In another embodiment, the therapeutically effective amount of Ca-AKG is at least about 600 mg. In another embodiment, the therapeutically effective amount of Ca-AKG is at least about 650 mg. In another embodiment, the therapeutically effective amount of Ca-AKG is at least about 700 mg. In another embodiment, the therapeutically effective amount of Ca-AKG is at least about 750 mg. In another embodiment, the therapeutically effective amount of Ca-AKG is at least about 800 mg. In another embodiment, the therapeutically effective amount of Ca-AKG is at least about 900 mg. In another embodiment, the therapeutically effective amount of Ca-AKG is at least about 1000 mg. In another embodiment, the therapeutically effective amount of Ca-AKG is at least about 1100 mg. In another embodiment, the therapeutically effective amount of Ca-AKG is at least about 1200 mg. In another embodiment, the therapeutically effective amount of Ca-AKG is at least about 1300 mg. In another embodiment, the therapeutically effective amount of Ca-AKG is at least about 1400 mg. In another embodiment, the therapeutically effective amount of Ca-AKG is at least about 1500 mg. In another embodiment, the therapeutically effective amount of Ca-AKG is at least about 1600 mg. In another embodiment, the therapeutically effective amount of Ca-AKG is at least about 1700 mg. In another embodiment, the therapeutically effective amount of Ca-AKG is at least about 1800 mg. In another embodiment, the therapeutically effective amount of Ca-AKG is at least about 1900 mg. In another embodiment, the therapeutically effective amount of Ca-AKG is at least about 2000 mg.

In another embodiment, the therapeutically effective amount of Ca-AKG is at least about 5 mg/kg. In another embodiment, the therapeutically effective amount of Ca-AKG is at least about 10 mg/kg. In another embodiment, the therapeutically effective amount of Ca-AKG is at least about 15 mg/kg. In another embodiment, the therapeutically effective amount of Ca-AKG is at least about 16 mg/kg. In another embodiment, the therapeutically effective amount of Ca-AKG is at least about 16.1 mg/kg. In another embodiment, the therapeutically effective amount of Ca-AKG is at least about 16.2 mg/kg. In another embodiment, the therapeutically effective amount of Ca-AKG is at least about 16.3 mg/kg. In another embodiment, the therapeutically effective amount of Ca-AKG is at least about 16.4 bmg/kg. In another embodiment, the therapeutically effective amount of Ca-AKG is at least about 16.5 mg/kg. In another embodiment, the therapeutically effective amount of Ca-AKG is at least about 16.6 mg/kg. In another embodiment, the therapeutically effective amount of Ca-AKG is at least about 16.7 mg/kg. In another embodiment, the therapeutically effective amount of Ca-AKG is at least about 16.8 mg/kg. In another embodiment, the therapeutically effective amount of Ca-AKG is at least about 16.9 mg/kg. In another embodiment, the therapeutically effective amount of Ca-AKG is at least about 17 mg/kg. In another embodiment, the therapeutically effective amount of Ca-AKG is at least about 18 mg/kg. In another embodiment, the therapeutically effective amount of Ca-AKG is at least about 19 mg/kg. In another embodiment, the therapeutically effective amount of Ca-AKG is at least about 20 mg/kg. In another embodiment, the therapeutically effective amount of Ca-AKG is at least about 21 mg/kg. In another embodiment, the therapeutically effective amount of Ca-AKG is at least about 22 mg/kg. In another embodiment, the therapeutically effective amount of Ca-AKG is at least about 23 mg/kg. In another embodiment, the therapeutically effective amount of Ca-AKG is at least about 24 mg/kg. In another embodiment, the therapeutically effective amount of Ca-AKG is at least about 25 mg/kg. In another embodiment, the therapeutically effective amount of Ca-AKG is at least about 28 mg/kg. In another embodiment, the therapeutically effective amount of Ca-AKG is at least about 30 mg/kg. In another embodiment, the therapeutically effective amount of Ca-AKG is at least about 33 mg/kg. In another embodiment, the therapeutically effective amount of Ca-AKG is at least about 35 mg/kg. In another embodiment, the therapeutically effective amount of Ca-AKG is at least 16.6 mg/kg.

In another embodiment, the therapeutically effective amount of vitamin B2 is about 1 mg to about 50 mg. In another embodiment, the therapeutically effective amount of vitamin B2 is about 2 mg to about 50 mg. In another embodiment, the therapeutically effective amount of vitamin B2 is about 3 mg to about 50 mg. In another embodiment, the therapeutically effective amount of vitamin B2 is about 4 mg to about 50 mg. In another embodiment, the therapeutically effective amount of vitamin B2 is about 5 mg to about 50 mg. In another embodiment, the therapeutically effective amount of vitamin B2 is about 7 mg to about 50 mg. In another embodiment, the therapeutically effective amount of vitamin B2 is about 10 mg to about 50 mg. In another embodiment, the therapeutically effective amount of vitamin B2 is about 12 mg to about 50 mg. In another embodiment, the therapeutically effective amount of vitamin B2 is about 15 mg to about 50 mg. In another embodiment, the therapeutically effective amount of vitamin B2 is about 17 mg to about 50 mg. In another embodiment, the therapeutically effective amount of vitamin B2 is about 20 mg to about 50 mg. In another embodiment, the therapeutically effective amount of vitamin B2 is about 25 mg to about 50 mg. In another embodiment, the therapeutically effective amount of vitamin B2 is about 30 mg to about 50 mg. In another embodiment, the therapeutically effective amount of vitamin B2 is about 35 mg to about 50 mg. In another embodiment, the therapeutically effective amount of vitamin B2 is about 40 mg to about 50 mg. In another embodiment, the therapeutically effective amount of vitamin B2 is about 45 mg to about 50 mg.

In another embodiment, the therapeutically effective amount of vitamin B2 is about 1 mg to about 25 mg. In another embodiment, the therapeutically effective amount of vitamin B2 is about 2 mg to about 25 mg. In another embodiment, the therapeutically effective amount of vitamin B2 is about 3 mg to about 25 mg. In another embodiment, the therapeutically effective amount of vitamin B2 is about 4 mg to about 25 mg. In another embodiment, the therapeutically effective amount of vitamin B2 is about 5 mg to about 25 mg. In another embodiment, the therapeutically effective amount of vitamin B2 is about 6 mg to about 25 mg. In another embodiment, the therapeutically effective amount of vitamin B2 is about 7 mg to about 25 mg. In another embodiment, the therapeutically effective amount of vitamin B2 is about 8 mg to about 25 mg. In another embodiment, the therapeutically effective amount of vitamin B2 is about 9 mg to about 25 mg. In another embodiment, the therapeutically effective amount of vitamin B2 is about 10 mg to about 25 mg. In another embodiment, the therapeutically effective amount of vitamin B2 is about 11 mg to about 25 mg. In another embodiment, the therapeutically effective amount of vitamin B2 is about 12 mg to about 25 mg. In another embodiment, the therapeutically effective amount of vitamin B2 is about 13 mg to about 25 mg. In another embodiment, the therapeutically effective amount of vitamin B2 is about 14 mg to about 25 mg. In another embodiment, the therapeutically effective amount of vitamin B2 is about 15 mg to about 25 mg. In another embodiment, the therapeutically effective amount of vitamin B2 is about 16 mg to about 25 mg. In another embodiment, the therapeutically effective amount of vitamin B2 is about 17 mg to about 25 mg. In another embodiment, the therapeutically effective amount of vitamin B2 is about 18 mg to about 25 mg. In another embodiment, the therapeutically effective amount of vitamin B2 is about 19 mg to about 25 mg. In another embodiment, the therapeutically effective amount of vitamin B2 is about 20 mg to about 25 mg. In another embodiment, the therapeutically effective amount of vitamin B2 is about 21 mg to about 25 mg. In another embodiment, the therapeutically effective amount of vitamin B2 is about 22 mg to about 25 mg. In another embodiment, the therapeutically effective amount of vitamin B2 is about 23 mg to about 25 mg. In another embodiment, the therapeutically effective amount of vitamin B2 is about 24 mg to about 25 mg.

In another embodiment, the therapeutically effective amount of vitamin B6 is about 1 mg to about 150 mg. In another embodiment, the therapeutically effective amount of vitamin B6 is about 2 mg to about 150 mg. In another embodiment, the therapeutically effective amount of vitamin B6 is about 3 mg to about 150 mg. In another embodiment, the therapeutically effective amount of vitamin B6 is about 4 mg to about 150 mg. In another embodiment, the therapeutically effective amount of vitamin B6 is about 5 mg to about 150 mg. In another embodiment, the therapeutically effective amount of vitamin B6 is about 7 mg to about 150 mg. In another embodiment, the therapeutically effective amount of vitamin B6 is about 10 mg to about 150 mg. In another embodiment, the therapeutically effective amount of vitamin B6 is about 12 mg to about 150 mg. In another embodiment, the therapeutically effective amount of vitamin B6 is about 15 mg to about 150 mg. In another embodiment, the therapeutically effective amount of vitamin B6 is about 17 mg to about 150 mg. In another embodiment, the therapeutically effective amount of vitamin B6 is about 20 mg to about 150 mg. In another embodiment, the therapeutically effective amount of vitamin B6 is about 25 mg to about 150 mg. In another embodiment, the therapeutically effective amount of vitamin B6 is about 30 mg to about 150 mg. In another embodiment, the therapeutically effective amount of vitamin B6 is about 35 mg to about 150 mg. In another embodiment, the therapeutically effective amount of vitamin B6 is about 40 mg to about 150 mg. In another embodiment, the therapeutically effective amount of vitamin B6 is about 45 mg to about 150 mg. In another embodiment, the therapeutically effective amount of vitamin B6 is about 50 mg to about 150 mg.

In another embodiment, the therapeutically effective amount of vitamin B6 is about 1 mg to about 100 mg. In another embodiment, the therapeutically effective amount of vitamin B6 is about 2 mg to about 100 mg. In another embodiment, the therapeutically effective amount of vitamin B6 is about 3 mg to about 100 mg. In another embodiment, the therapeutically effective amount of vitamin B6 is about 4 mg to about 100 mg. In another embodiment, the therapeutically effective amount of vitamin B6 is about 5 mg to about 100 mg. In another embodiment, the therapeutically effective amount of vitamin B6 is about 7 mg to about 100 mg. In another embodiment, the therapeutically effective amount of vitamin B6 is about 10 mg to about 100 mg. In another embodiment, the therapeutically effective amount of vitamin B6 is about 12 mg to about 100 mg. In another embodiment, the therapeutically effective amount of vitamin B6 is about 15 mg to about 100 mg. In another embodiment, the therapeutically effective amount of vitamin B6 is about 17 mg to about 100 mg. In another embodiment, the therapeutically effective amount of vitamin B6 is about 20 mg to about 100 mg. In another embodiment, the therapeutically effective amount of vitamin B6 is about 25 mg to about 100 mg. In another embodiment, the therapeutically effective amount of vitamin B6 is about 30 mg to about 100 mg. In another embodiment, the therapeutically effective amount of vitamin B6 is about 35 mg to about 100 mg. In another embodiment, the therapeutically effective amount of vitamin B6 is about 40 mg to about 100 mg. In another embodiment, the therapeutically effective amount of vitamin B6 is about 45 mg to about 100 mg. In another embodiment, the therapeutically effective amount of vitamin B6 is about 50 mg to about 100 mg.

In another embodiment, the therapeutically effective amount of one or more folates (vitamin B9) is about 100 μg to about 1000 μg. In another embodiment, the therapeutically effective amount of one or more folates (vitamin B9) is about 150 μg to about 1000 μg. In another embodiment, the therapeutically effective amount of one or more folates (vitamin B9) is about 200 μg to about 1000 μg. In another embodiment, the therapeutically effective amount of one or more folates (vitamin B9) is about 250 μg to about 1000 μg. In another embodiment, the therapeutically effective amount of one or more folates (vitamin B9) is about 300 μg to about 1000 μg. In another embodiment, the therapeutically effective amount of one or more folates (vitamin B9) is about 350 μg to about 1000 μg. In another embodiment, the therapeutically effective amount of one or more folates (vitamin B9) is about 400 μg to about 1000 μg. In another embodiment, the therapeutically effective amount of one or more folates (vitamin B9) is about 450 μg to about 1000 μg. In another embodiment, the therapeutically effective amount of one or more folates (vitamin B9) is about 500 μg to about 1000 μg. In another embodiment, the therapeutically effective amount of one or more folates (vitamin B9) is about 550 μg to about 1000 μg. In another embodiment, the therapeutically effective amount of one or more folates (vitamin B9) is about 600 μg to about 1000 μg. In another embodiment, the therapeutically effective amount of one or more folates (vitamin B9) is about 650 μg to about 1000 μg. In another embodiment, the therapeutically effective amount of one or more folates (vitamin B9) is about 700 μg to about 1000 μg. In another embodiment, the therapeutically effective amount of one or more folates (vitamin B9) is about 800 μg to about 1000 μg. In another embodiment, the therapeutically effective amount of one or more folates (vitamin B9) is about 900 μg to about 1000 μg.

In another embodiment, the therapeutically effective amount of one or more folates (vitamin B9) is about 100 μg to about 1200 μg. In another embodiment, the therapeutically effective amount of one or more folates (vitamin B9) is about 150 μg to about 1200 μg. In another embodiment, the therapeutically effective amount of one or more folates (vitamin B9) is about 200 μg to about 1200 μg. In another embodiment, the therapeutically effective amount of one or more folates (vitamin B9) is about 250 μg to about 1200 μg. In another embodiment, the therapeutically effective amount of one or more folates (vitamin B9) is about 300 μg to about 1200 μg. In another embodiment, the therapeutically effective amount of one or more folates (vitamin B9) is about 350 μg to about 1200 μg. In another embodiment, the therapeutically effective amount of one or more folates (vitamin B9) is about 400 μg to about 1200 μg. In another embodiment, the therapeutically effective amount of one or more folates (vitamin B9) is about 450 μg to about 1200 μg. In another embodiment, the therapeutically effective amount of one or more folates (vitamin B9) is about 500 μg to about 1200 μg. In another embodiment, the therapeutically effective amount of one or more folates (vitamin B9) is about 550 μg to about 1200 μg. In another embodiment, the therapeutically effective amount of one or more folates (vitamin B9) is about 600 μg to about 1200 μg. In another embodiment, the therapeutically effective amount of one or more folates (vitamin B9) is about 650 μg to about 1200 μg. In another embodiment, the therapeutically effective amount of one or more folates (vitamin B9) is about 700 μg to about 1200 μg. In another embodiment, the therapeutically effective amount of one or more folates (vitamin B9) is about 800 μg to about 1200 μg. In another embodiment, the therapeutically effective amount of one or more folates (vitamin B9) is about 900 μg to about 1200 μg. In another embodiment, the therapeutically effective amount of one or more folates (vitamin B9) is about 1200 μg to about 1200 μg. In another embodiment, the therapeutically effective amount of one or more folates (vitamin B9) is about 1100 μg to about 1200 μg.

In another embodiment, the therapeutically effective amount of one or more folates (vitamin B9) is about 100 μg to about 2000 μg. In another embodiment, the therapeutically effective amount of one or more folates (vitamin B9) is about 150 μg to about 2000 μg. In another embodiment, the therapeutically effective amount of one or more folates (vitamin B9) is about 200 μg to about 2000 μg. In another embodiment, the therapeutically effective amount of one or more folates (vitamin B9) is about 250 μg to about 2000 μg. In another embodiment, the therapeutically effective amount of one or more folates (vitamin B9) is about 300 μg to about 2000 μg. In another embodiment, the therapeutically effective amount of one or more folates (vitamin B9) is about 350 μg to about 2000 μg. In another embodiment, the therapeutically effective amount of one or more folates (vitamin B9) is about 400 μg to about 2000 μg. In another embodiment, the therapeutically effective amount of one or more folates (vitamin B9) is about 450 μg to about 2000 μg. In another embodiment, the therapeutically effective amount of one or more folates (vitamin B9) is about 500 μg to about 2000 μg. In another embodiment, the therapeutically effective amount of one or more folates (vitamin B9) is about 550 μg to about 2000 μg. In another embodiment, the therapeutically effective amount of one or more folates (vitamin B9) is about 600 μg to about 2000 μg. In another embodiment, the therapeutically effective amount of one or more folates (vitamin B9) is about 650 μg to about 2000 μg. In another embodiment, the therapeutically effective amount of one or more folates (vitamin B9) is about 700 μg to about 2000 μg. In another embodiment, the therapeutically effective amount of one or more folates (vitamin B9) is about 800 μg to about 2000 μg. In another embodiment, the therapeutically effective amount of one or more folates (vitamin B9) is about 900 μg to about 2000 μg. In another embodiment, the therapeutically effective amount of one or more folates (vitamin B9) is about 1000 μg to about 2000 μg. In another embodiment, the therapeutically effective amount of one or more folates (vitamin B9) is about 1100 μg to about 2000 μg. In another embodiment, the therapeutically effective amount of one or more folates (vitamin B9) is about 1200 μg to about 2000 μg. In another embodiment, the therapeutically effective amount of one or more folates (vitamin B9) is about 1300 μg to about 2000 μg. In another embodiment, the therapeutically effective amount of one or more folates (vitamin B9) is about 1400 μg to about 2000 μg. In another embodiment, the therapeutically effective amount of one or more folates (vitamin B9) is about 1500 μg to about 2000 μg. In another embodiment, the therapeutically effective amount of one or more folates (vitamin B9) is about 1600 μg to about 2000 μg. In another embodiment, the therapeutically effective amount of one or more folates (vitamin B9) is about 1700 μg to about 2000 μg. In another embodiment, the therapeutically effective amount of one or more folates (vitamin B9) is about 1800 μg to about 2000 μg. In another embodiment, the therapeutically effective amount of one or more folates (vitamin B9) is about 1900 μg to about 2000 μg.

In another embodiment, the therapeutically effective amount of one or more folates (vitamin B9) is about 400 μg to about 4000 μg. In another embodiment, the therapeutically effective amount of one or more folates (vitamin B9) is about 800 μg to about 4000 μg. In another embodiment, the therapeutically effective amount of one or more folates (vitamin B9) is about 1200 μg to about 4000 μg. In another embodiment, the therapeutically effective amount of one or more folates (vitamin B9) is about 1600 μg to about 4000 μg. In another embodiment, the therapeutically effective amount of one or more folates (vitamin B9) is about 2000 μg to about 4000 μg. In another embodiment, the therapeutically effective amount of one or more folates (vitamin B9) is about 2400 μg to about 4000 μg. In another embodiment, the therapeutically effective amount of one or more folates (vitamin B9) is about 2800 μg to about 4000 μg. In another embodiment, the therapeutically effective amount of one or more folates (vitamin B9) is about 3200 μg to about 4000 μg. In another embodiment, the therapeutically effective amount of one or more folates (vitamin B9) is about 3600 μg to about 4000 μg.

In another embodiment, the therapeutically effective amount of one or more folates (vitamin B9) is about 400 μg to about 5000 μg. In another embodiment, the therapeutically effective amount of one or more folates (vitamin B9) is about 800 μg to about 5000 μg. In another embodiment, the therapeutically effective amount of one or more folates (vitamin B9) is about 1200 μg to about 5000 μg. In another embodiment, the therapeutically effective amount of one or more folates (vitamin B9) is about 1600 μg to about 5000 μg. In another embodiment, the therapeutically effective amount of one or more folates (vitamin B9) is about 2000 μg to about 5000 μg. In another embodiment, the therapeutically effective amount of one or more folates (vitamin B9) is about 2400 μg to about 5000 μg. In another embodiment, the therapeutically effective amount of one or more folates (vitamin B9) is about 2800 μg to about 5000 μg. In another embodiment, the therapeutically effective amount of one or more folates (vitamin B9) is about 3200 μg to about 5000 μg. In another embodiment, the therapeutically effective amount of one or more folates (vitamin B9) is about 3600 μg to about 5000 μg. In another embodiment, the therapeutically effective amount of one or more folates (vitamin B9) is about 4000 μg to about 5000 μg. In another embodiment, the therapeutically effective amount of one or more folates (vitamin B9) is about 4400 μg to about 5000 μg.

In certain embodiments, methods and compositions with higher folate amounts, such as upper limits of 4000 μg or 5000 μg are described. It is understood herein that women who have had or are at risk of having a pregnancy affected by a neural tube defect may be administered higher amounts of folates. It is also understood herein women who are considering or deciding to become pregnant may be prophylactically administered higher amounts of folates.

It is to be understood that the methods and compositions described herein can include a single folate or and any mixtures of folates. Illustrative folates include folic acid, pteroyl acid amides, such as pteroyl acid amides of glutamate and pteroyl acid amides of polyglutamate, methyl folates of the foregoing, tetrahydro folates of the foregoing, and methyltetrahydrofolates of the foregoing, such as 5-methylfolates and 5-methyltetrahydrofolates.

In another embodiment, the therapeutically effective amount of vitamin B12 is about 100 μg to about 2000 μg. In another embodiment, the therapeutically effective amount of vitamin B12 is about 150 μg to about 2000 μg. In another embodiment, the therapeutically effective amount of vitamin B12 is about 200 μg to about 2000 μg. In another embodiment, the therapeutically effective amount of vitamin B12 is about 250 μg to about 2000 μg. In another embodiment, the therapeutically effective amount of vitamin B12 is about 300 μg to about 2000 μg. In another embodiment, the therapeutically effective amount of vitamin B12 is about 350 μg to about 2000 μg. In another embodiment, the therapeutically effective amount of vitamin B12 is about 400 μg to about 2000 μg. In another embodiment, the therapeutically effective amount of vitamin B12 is about 450 μg to about 2000 μg. In another embodiment, the therapeutically effective amount of vitamin B12 is about 500 μg to about 2000 μg. In another embodiment, the therapeutically effective amount of vitamin B12 is about 550 μg to about 2000 μg. In another embodiment, the therapeutically effective amount of vitamin B12 is about 600 μg to about 2000 μg. In another embodiment, the therapeutically effective amount of vitamin B12 is about 650 μg to about 2000 μg. In another embodiment, the therapeutically effective amount of vitamin B12 is about 700 μg to about 2000 μg. In another embodiment, the therapeutically effective amount of vitamin B12 is about 800 μg to about 2000 μg. In another embodiment, the therapeutically effective amount of vitamin B12 is about 900 μg to about 2000 μg. In another embodiment, the therapeutically effective amount of vitamin B12 is about 1000 μg to about 2000 μg.

In another embodiment, the therapeutically effective amount of vitamin B12 is about 100 μg to about 1500 μg. In another embodiment, the therapeutically effective amount of vitamin B12 is about 200 μg to about 1500 μg. In another embodiment, the therapeutically effective amount of vitamin B12 is about 300 μg to about 1500 μg. In another embodiment, the therapeutically effective amount of vitamin B12 is about 400 μg to about 1500 μg. In another embodiment, the therapeutically effective amount of vitamin B12 is about 500 μg to about 1500 μg. In another embodiment, the therapeutically effective amount of vitamin B12 is about 600 μg to about 1500 μg. In another embodiment, the therapeutically effective amount of vitamin B12 is about 700 μg to about 1500 μg. In another embodiment, the therapeutically effective amount of vitamin B12 is about 800 μg to about 1500 μg. In another embodiment, the therapeutically effective amount of vitamin B12 is about 900 μg to about 1500 μg. In another embodiment, the therapeutically effective amount of vitamin B12 is about 1000 μg to about 1500 μg.

In another embodiment, the therapeutically effective amount of Ca-AKG is at least 16.6 mg/kg. In another embodiment, the composition further comprises vitamin A. In another embodiment, the composition comprises 450 mcg of vitamin A.

In another embodiment, the composition further comprises vitamin D3. In another embodiment, the composition comprises 12.5 mcg (500 IU) of vitamin D3.

Disclosed herein, in another embodiment, are compositions comprising: calcium alpha-ketoglutarate, and one or more agents selected from the group consisting of surfactants, preservatives, flavoring agents, vitamins, antioxidants, sweetening agents, and combinations thereof. In another embodiment, the amount of calcium alpha-ketoglutarate is from 50 mg to 5000 mg. In another embodiment, the amount of calcium alpha-ketoglutarate is from 100 mg to 2000 mg. In another embodiment, the amount of calcium alpha-ketoglutarate is about 250 mg.

In another embodiment, the amount of calcium alpha-ketoglutarate is about 500 mg. In another embodiment, the amount of calcium alpha-ketoglutarate is about 750 mg. In another embodiment, the composition comprises a vitamin. In another embodiment, the vitamin is vitamin A. In another embodiment, the amount of vitamin A is from 100 mcg to 3000 mcg. In another embodiment, the amount of vitamin A is from 200 mcg to 1000 mcg. In another embodiment, the amount of vitamin A is about 250 mcg. In another embodiment, the amount of vitamin A is about 450 mcg. In another embodiment, the amount of vitamin A is about 650 mcg. In another embodiment, the vitamin A is retinyl palmitate. In another embodiment, the vitamin is vitamin D. In another embodiment, the amount of vitamin D is from 50 IU to 3000 IU. In another embodiment, the amount of vitamin D is from 200 IU to 2000 IU. In another embodiment, the amount of vitamin D is about 250 IU. In another embodiment, the amount of vitamin D is about 500 IU. In another embodiment, the amount of vitamin D is about 750 IU. In another embodiment, the vitamin D is cholecalciferol. In another embodiment, the composition further comprises a sweetener. In another embodiment, the sweetener is isomalt. In another embodiment, the composition further comprises wax. In another embodiment, the wax is carnauba wax and/or rice bran wax. In another embodiment, the composition further comprises one or more excipients. In another embodiment, the excipient is selected from the group consisting of solubilizing agents, diluents, fillers, disintegrants, lubricants, glidants, binders, suspending agents, and combinations thereof. In another embodiment, the excipient is selected from the group consisting of solubilizing agents, diluents, fillers, disintegrants, lubricants, glidants, and combinations thereof. In another embodiment, the composition further comprises a first lubricant. In another embodiment, the first lubricant is stearic acid. In another embodiment, the composition comprises a second lubricant. In another embodiment, the second lubricant is magnesium stearate. In another embodiment, the composition comprises a glidant. In another embodiment, the glidant is silica. In another embodiment, the calcium alpha-ketoglutarate is calcium alpha-ketoglutarate monohydrate.

In another embodiment, the composition is formulated into an orally administered form. In another embodiment, the orally administered form comprises a tablet, a powder, a suspension, a serum, an emulsion, a capsule, a granule, a pill, a gel, a solution, or a syrup. In another embodiment, the orally administered form is a sustained release dosage form. In another embodiment, the orally administered form is formulated into animal feed. In another embodiment, the therapeutically effective amount of Ca-AKG in animal feed is at least about 0.025% w/w to at least about 10% w/w. In another embodiment, the orally administered form is the gel. In another embodiment, the composition is formulated into an injectable administered form. In another embodiment, the injectable administered form comprises a liquid, a suspension, or a solution. In another embodiment, the injectable administered form is a sustained release dosage form. In another embodiment, the composition is formulated into a topically administered form. In another embodiment, the topically administered form is a cream, a foam, a gel, a lotion, an ointment, or a serum. In another embodiment, the composition is formulated into a hair care product. In another embodiment, the hair care product is a shampoo, a conditioner, hair spray, or a moisturizer. In another embodiment, the composition further comprises a pharmaceutically acceptable excipient selected from the group consisting of an anti-adherent, a binder, a coating, a color, disintegrant, a flavor, a glidant, a lubricant, a preservative, a sorbent, or a vehicle. In another embodiment, the subject is pre-screened for plasma AKG level. In another embodiment, the subject has reduced plasma AKG level. In another embodiment, the subject has at least 6-fold, at least 8-fold, or at least 10-fold reduction in plasma AKG level compared to plasma AKG level in a control sample. In another embodiment, the control sample is a serum sample from a control subject who is at least 2, at least 3, at least 4, at least 5, at least 8, or at least 10 years younger than the subject.

In another embodiment, the subject is pre-screened for a DNA methylation profile. In another embodiment, the DNA methylation profile of the subject is comparable to a DNA methylation profile of a second control subject. In another embodiment, the control subject is at least of same age as the subject. In another embodiment, the composition alters the DNA methylation profile of the subject. In another embodiment, the altered DNA methylation profile of the subject is comparable to the second control subject who is at least 2, at least 3, at least 4, at least 5, at least 8, or at least 10 years younger than the subject. In another embodiment, the composition further delays or reverses an age-related phenotype. In another embodiment, the age-related phenotype comprises osteoporosis, obesity, type-2 diabetes, macular degeneration, or an autoimmune disease. In another embodiment, the age-related phenotype comprises sarcopenia. In another embodiment, the age-related phenotype comprises impaired cognition. In another embodiment, the age-related phenotype comprises decreased stem cell production. In another embodiment, the age-related phenotype comprises increased inflammation. In another embodiment, the age-related phenotype comprises age-related epigenetic changes. In another embodiment, epigenetic changes comprises changes in DNA methylation profile. In another embodiment, the composition further induces or promotes collagen synthesis. In another embodiment, the composition further enhances adult stem cell function.

As used herein, the term increased longevity generally refers to the subject living longer than or beyond the corresponding life expectancy. For example, life expectancy is a statistical average number of years remaining for the subject at a specific age.

It is to be understood herein that the compounds, compositions, unit doses, and methods described herein may be used alone or in combination with other compounds useful for treating age-related illness and/or increasing longevity, including those compounds that may be therapeutically effective by the same or different modes of action. In addition, it is to be understood herein that the compounds described herein may be used in combination with other compounds that are administered to treat other symptoms of age-related illness.

In each of the foregoing and each of the following embodiments, unless otherwise indicated, it is to be understood that the formulae include and represent not only all pharmaceutically acceptable salts of the compounds, but also include any and all hydrates and/or solvates of the compound formulae. It is appreciated that certain functional groups, such as the hydroxy, amino, and like groups form complexes and/or coordination compounds with water and/or various solvents, in the various physical forms of the compounds. Accordingly, the above formulae are to be understood to be a description of such hydrates and/or solvates, including pharmaceutically acceptable solvates.

In another embodiment, the compounds described herein are formulated in oral dosage forms. Illustrative formats for oral administration include tablets, powders, granules, pills, dragees, capsules, liquids, serums, gels, solutions, syrups, elixirs, slurries, suspensions, emulsions, and the like.

In making the pharmaceutical compositions of the compounds described herein, a therapeutically effective amount of one or more compounds in any of the various forms described herein may be mixed with one or more excipients, diluted by one or more excipients, or enclosed within such a carrier which can be in the form of a capsule, sachet, paper, or other container. Excipients may serve as a diluent, and can be solid, semi-solid, or liquid materials, which act as a vehicle, carrier or medium for the active ingredient. Thus, the formulation compositions can be in the form of tablets, pills, powders, lozenges, sachets, cachets, elixirs, suspensions, emulsions, solutions, syrups, aerosols (as a solid or in a liquid medium), ointments, soft and hard gelatin capsules, suppositories, sterile injectable solutions, and sterile packaged powders. The compositions may contain anywhere from about 0.1% to about 99.9% active ingredients, depending upon the selected dose and dosage form.

Illustrative examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include: lubricating agents such as talc, magnesium stearate, and mineral oil; wetting agents; emulsifying and suspending agents; preserving agents such as methyl hydroxybenzoate and/or propyl hydroxybenzoate; sweetening agents; and flavoring agents. The compositions can be formulated so as to provide quick, sustained or delayed release of the active ingredient after administration to the host animal by employing procedures known in the art. It is to be understood that one or more carriers, one or more diluents, one or more excipients, and combinations of the foregoing may be used in making the pharmaceutical compositions described herein. It is appreciated that the carriers, diluents, and excipients used to prepare the compositions described herein are advantageously GRAS (generally regarded as safe) compounds. It is also appreciated that acids and bases used to make salts, as described herein, and/or solvents used to make solvates, as described herein, are also advantageously GRAS compounds.

Illustrative examples of emulsifying agents include naturally occurring gums (e.g., gum acacia or gum tragacanth) and naturally occurring phosphatides (e.g., soybean lecithin and sorbitan monooleate derivatives). Examples of antioxidants are butylated hydroxy anisole (BHA), ascorbic acid and derivatives thereof, tocopherol and derivatives thereof, butylated hydroxy anisole, and cysteine. Examples of preservatives are parabens, such as methyl or propyl p-hydroxybenzoate, and benzalkonium chloride. Examples of humectants are glycerin, propylene glycol, sorbitol, and urea. Examples of penetration enhancers are propylene glycol, DMSO, triethanolamine, N,N-dimethylacetamide, N,N-dimethylformamide, 2-pyrrolidone and derivatives thereof, tetrahydrofurfuryl alcohol, and AZONE. Examples of chelating agents are sodium EDTA, citric acid, and phosphoric acid. Examples of gel forming agents are CARBOPOL, cellulose derivatives, bentonite, alginates, gelatin, and polyvinylpyrrolidone. Examples of ointment bases are beeswax, paraffin, cetyl palmitate, vegetable oils, sorbitan esters of fatty acids (Span), polyethylene glycols, and condensation products between sorbitan esters of fatty acids and ethylene oxide (e.g., polyoxyethylene sorbitan monooleate (TWEEN)).

Solid Dosage Forms for Oral Use. Formulations for oral use include tablets containing the active ingredient(s) in a mixture with non-toxic pharmaceutically acceptable excipients. These excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like.

The solid tablet compositions may include a coating adapted to protect the composition from unwanted chemical changes, (e.g., chemical degradation prior to the release of the active drug substance). The coating may be applied on the solid dosage form in a similar manner as that described in Encyclopedia of Pharmaceutical Technology.

The tablets may be uncoated or they may be coated by known techniques, optionally to delay disintegration and absorption in the gastrointestinal tract and thereby providing a sustained action over a longer period. The coating may be adapted to release the active drug substances in a predetermined pattern (e.g., in order to achieve a controlled release formulation) or it may be adapted not to release the active drug substances until after passage of the stomach (enteric coating). The coating may be a sugar coating, a film coating (e.g., based on hydroxypropyl methylcellulose, methylcellulose, methyl hydroxyethylcellulose, hydroxypropylcellulose, carboxymethylcellulose, acrylate copolymers, polyethylene glycols, and/or polyvinylpyrrolidone), or an enteric coating (e.g., based on methacrylic acid copolymer, cellulose acetate phthalate, hydroxypropyl methylcellulose phthalate, hydroxypropyl methylcellulose acetate succinate, polyvinyl acetate phthalate, shellac, and/or ethylcellulose). Furthermore, a time delay material such as, e.g., glyceryl monostearate or glyceryl distearate may be employed.

Tablets described herein may comprise the active drug substances included in a controlled-release matrix to delay dissolution and/or disintegration in the stomach. Illustratively, the tablets comprise one or more hydroxypropylmethylcelluloses (HPMC), including one or more HPMC selected from the group consisting of HPMC with number average molecular weight of about 22,000 to about 30,000, about 68,000 to about 95,000, about 115,000 to about 150,000, and about 220,000 to about 300,000, and combinations thereof.

In another embodiment, tablets described herein comprise HPMC with number average molecular weight of about 68,000-95,000. In another embodiment, the tablets comprise HPMC with number average molecular weight of about 115,000-150,000. In another embodiment, the tablets comprise a mixture of HPMC with number average molecular weight about 68,000-95,000 and about 115,000-150,000.

In another embodiment, tablets described herein are characterized by a release rate of the calcium AKG in a conventional USP 24 (Paddle Apparatus 2) operating at 100 rpm in a 0.1 N HCl medium of about 15%, about 20% or less, about 25% or less, about 30% or less, or about 35% or less after about 1 hour.

In another embodiment, tablets described herein are characterized by a release rate of the calcium AKG in a conventional USP 24 (Paddle Apparatus 2) operating at 100 rpm in a 0.1 N HCl medium of about 40%, about 45% or less, about 50% or less, about 55% or less, or about 60% or less after about 3 hours.

In another embodiment, tablets described herein are characterized by a release rate of the calcium AKG in a conventional USP 24 (Paddle Apparatus 2) operating at 100 rpm in a 0.1 N HCl medium of about 65%, about 70% or less, about 75% or less, about 80% or less, or about 85% or less after about 6 hours.

In another embodiment, tablets described herein are characterized by a release rate of the calcium AKG in a conventional USP 24 (Paddle Apparatus 2) operating at 100 rpm in a 0.1 N HCl medium of at least about 90% (i.e. about 90% or greater) after about 9 hours.

In another embodiment, compositions comprising 500 mg of calcium alpha-ketoglutarate monohydrate; one or more B vitamins; and further comprising isomalt, vegetable wax (carnauba and/or rice bran), stearic acid, magnesium stearate, and silica are described herein. Illustratively, the composition is a unit dose configured for oral administration. Illustratively, the composition or unit dose includes one or more B vitamins selected from 50 mg vitamin B6, 500 μg folate (vitamin B9), and 500 μg vitamin B12, and combinations thereof.

In another embodiment, compositions comprising 500 mg of calcium alpha-ketoglutarate monohydrate; one or more B vitamins; and further comprising isomalt, vegetable wax (carnauba and/or rice bran), stearic acid, magnesium stearate, and silica are described herein. Illustratively, the composition is a unit dose configured for oral administration. Illustratively, the composition or unit dose includes one or more B vitamins selected from 12.5 mg vitamin B2, 50 mg vitamin B6, 500 μg folate (vitamin B9), and 500 μg vitamin B12, and combinations thereof.

In another embodiment, compositions comprising 500 mg of calcium alpha-ketoglutarate monohydrate; one or more B vitamins; 450 mcg of retinyl palmitate; and further comprising isomalt, vegetable wax (carnauba and/or rice bran), stearic acid, magnesium stearate, and silica are described herein. Illustratively, the composition is a unit dose configured for oral administration. Illustratively, the composition or unit dose includes one or more B vitamins selected from 50 mg vitamin B6, 500 μg folate (vitamin B9), and 500 μg vitamin B12, and combinations thereof.

In another embodiment, compositions comprising 500 mg of calcium alpha-ketoglutarate monohydrate; one or more B vitamins; 450 mcg of retinyl palmitate; and further comprising isomalt, vegetable wax (carnauba and/or rice bran), stearic acid, magnesium stearate, and silica are described herein. Illustratively, the composition is a unit dose configured for oral administration. Illustratively, the composition or unit dose includes one or more B vitamins selected from 12.5 mg vitamin B2, 50 mg vitamin B6, 500 μg folate (vitamin B9), and 500 μg vitamin B12, and combinations thereof.

In another embodiment, compositions comprising 500 mg of calcium alpha-ketoglutarate monohydrate; one or more B vitamins; 12.5 mcg (500 IU) of cholecalciferol; and further comprising isomalt, vegetable wax (carnauba and/or rice bran), stearic acid, magnesium stearate, and silica are described herein. Illustratively, the composition is a unit dose configured for oral administration. Illustratively, the composition or unit dose includes one or more B vitamins selected from 50 mg vitamin B6, 500 μg folate (vitamin B9), and 500 μg vitamin B12, and combinations thereof.

In another embodiment, compositions comprising 500 mg of calcium alpha-ketoglutarate monohydrate; one or more B vitamins; 12.5 mcg (500 IU) of cholecalciferol; and further comprising isomalt, vegetable wax (carnauba and/or rice bran), stearic acid, magnesium stearate, and silica are described herein. Illustratively, the composition is a unit dose configured for oral administration. Illustratively, the composition or unit dose includes one or more B vitamins selected from 12.5 mg vitamin B2, 50 mg vitamin B6, 500 μg folate (vitamin B9), and 500 μg vitamin B12, and combinations thereof.

Controlled Release Oral Dosage Forms. Controlled release compositions for oral use may, e.g., be constructed to release the active drug by controlling the dissolution and/or the diffusion of the active drug substance. Illustrative sustained release formulations are described in U.S. Pat. Nos. 3,847,770; 3,916,899; 3,536,809; 3,598,123; 3,630,200; 4,008,719; 4,687,610; 4,769,027; 5,674,533; 5,059,595; 5,591,767; 5,120,548; 5,073,543; 5,639,476; 5,354,566; 5,733,566; and WO 2020/252014, the disclosures of which are incorporated herein, each in its entirety, by reference.

Dissolution or diffusion-controlled release can be achieved by appropriate coating of a tablet, capsule, pellet, or granulate formulation of compounds, or by incorporating the compound into an appropriate matrix. A controlled release coating may include one or more of the coating substances mentioned above and/or, e.g., shellac, beeswax, glycowax, castor wax, carnauba wax, stearyl alcohol, glyceryl monostearate, glyceryl distearate, glycerol palmitostearate, ethylcellulose, acrylic resins, dl-polylactic acid, cellulose acetate butyrate, polyvinyl chloride, polyvinyl acetate, vinyl pyrrolidone, polyethylene, polymethacrylate, methylmethacrylate, 2-hydroxymethacrylate, methacrylate hydrogels, 1,3 butylene glycol, ethylene glycol methacrylate, and/or polyethylene glycols. In a controlled release matrix formulation, the matrix material may also include, e.g., hydrated metylcellulose, carnauba wax, and stearyl alcohol, carbopol 934, silicone, glyceryl tristearate, methyl acrylate-methyl methacrylate, polyvinyl chloride, polyethylene, and/or halogenated fluorocarbon.

A controlled release composition containing one or more of the compounds of the claimed combinations may also be in the form of a buoyant tablet or capsule (i.e., a tablet or capsule that, upon oral administration, floats on top of the gastric content for a certain period of time). A buoyant tablet formulation of the compound(s) can be prepared by granulating a mixture of the drug(s) with excipients and 20-75% w/w of hydrocolloids, such as hydroxyethylcellulose, hydroxypropylcellulose, or hydroxypropylmethylcellulose. The obtained granules can then be compressed into tablets. On contact with the gastric juice, the tablet forms a substantially water-impermeable gel barrier around its surface. This gel barrier takes part in maintaining a density of less than one, thereby allowing the tablet to remain buoyant in the gastric juice.

Liquids for Oral Administration. Powders, dispersible powders, or granules suitable for preparation of an aqueous suspension by addition of water are convenient dosage forms for oral administration. Formulation as a suspension provides the active ingredient in a mixture with a dispersing or wetting agent, suspending agent, and one or more preservatives. Suitable dispersing or wetting agents are, for example, naturally occurring phosphatides (e.g., lecithin or condensation products of ethylene oxide with a fatty acid, a long chain aliphatic alcohol, or a partial ester derived from fatty acids) and a hexitol or a hexitol anhydride (e.g., polyoxyethylene stearate, polyoxyethylene sorbitol monooleate, polyoxyethylene sorbitan monooleate, and the like). Suitable suspending agents are, for example, sodium carboxymethylcellulose, methylcellulose, sodium alginate, and the like.

In another embodiment, therapeutically effective amounts of the active agents described herein are formulated into other solid oral dosage forms. Oral dosage forms include push fit capsules made of gelatin, as well as soft, sealed capsules made of gelatin and a plasticizer, such as glycerol or sorbitol. In specific embodiments, push fit capsules contain the active ingredients in admixture with one or more filler. Fillers include, by way of example only, lactose, binders such as starches, and/or lubricants such as talc or magnesium stearate, and optionally, stabilizers. In other embodiments, soft capsules, contain one or more active compound that is dissolved or suspended in a suitable liquid. Suitable liquids include, by way of example only, one or more fatty oil, liquid paraffin, or liquid polyethylene glycol. In addition, stabilizers are optionally added. In another embodiment, AKG or Ca-AKG is formulated into a soft gel capsule.

In another embodiment, AKG or CaAKG are formulated as coated beads as described in Patel, R R and Patel J K, “Novel Technologies of Oral Controlled Release Drug Delivery System,” Systematic Reviews in Pharmacy, July-December 2010, Vol. 1 (2), 128-132.

In another embodiment, AKG is formulated with fish oil in a gel cap. In another embodiment, AKG or CaAKG is formulated into an amino acid supplement including one or more of the following amino acids, L-histidine, L-isoleucine, L-leucine, L-lysine, L-methionine, L-phenylalanine, L-threonine, L-valine. In another embodiment, AKG or CaAKG is added as a source of glutamic acid.

In other embodiments, active agents described herein are formulated into oral liquid dosage forms. Exemplary liquid preparations for oral use include solutions, emulsions, serums, solutions, syrups or suspensions containing one or more active ingredients in a suitable vehicle. Syrups are clear viscous oral liquids containing high concentrations of sugar or other sweetening agents, in which active agents are solubilized in a pharmaceutically acceptable vehicle. Suspensions consist of finely divided particles of active agent suspended in pharmaceutically acceptable vehicle in which the particles are poorly soluble. Oral emulsions contain liquid forms of active agents dispersed as droplets in a continuous phase of another immiscible vehicle with the help of emulsifying agents (e.g. carbohydrates, gelatin, high molecular weight alcohols, wetting agents, colloidal clays, and the like).

In another embodiment, active agents are formulated into semi-solid oral dosage forms such as gels. Gels or jelly-like formulations have particular relevance for elderly or dysphagic patients with difficulty consuming other oral dosage forms. Gels are formed by adding active agents to water, adding a low critical concentration (e.g. 0.5-2.5%) of a gelling agent, heating, and cooling. Examples of suitable gelling agents include agar, gelatin, carrageenan, sodium caseinate, glycerogelatin, silk fibroin, gellan gum, kelcogel, xyloglucan, gellan, and pectin.

In another embodiment, the one or more active agents are formulated into a food product. In another embodiment, the food product is a drink for oral administration. Non-limiting examples of a suitable drink include fruit juice, a fruit drink, an artificially flavored drink, an artificially sweetened drink, a carbonated beverage, a sports drink, a liquid dairy product, a shake, a smoothie, or a caffeinated beverage. In another embodiment, the one or more active agents are a powder for constitution in the drink for oral administration.

In another embodiment, the food product is a solid foodstuff for oral administration. Suitable examples of a solid foodstuff include without limitation a food bar, a snack bar, a jelly bean, sports gel, a gummy, a sports chew, a cookie, a brownie, a muffin, a cracker, an ice cream bar, or a frozen yogurt bar.

In another embodiment, the food product is a sports drink. In another embodiment, the sports drink contains the active agents and sports drinks ingredients, including but not limited to, filtered water, brominated vegetable oil, high fructose corn syrup, citric acid, fruit juice, salt, sodium citrate, monopotassium phosphate, glycerol ester of wood rosin, and artificial colors.

In another embodiment, the food product is a smoothie. In another embodiment, the smoothie contains the active agents and smoothie ingredients, including but not limited to, milk (cow's milk, soy milk, or almond milk), yogurt, nut butter (peanut, almond or sun butter), honey, and ice. In another embodiment, the smoothie contains frozen mixed berries.

In another embodiment, the food product is a food bar. In another embodiment, the food bar contains the active agents and food bar ingredients, including but not limited to brown rice syrup, rolled oats, soy protein isolate, cane syrup, roasted soybeans, rice flour, dried cane syrup, unsweetened chocolate, soy flour, oat fiber, high oleic sunflower oil, cocoa butter, barley malt extract, sea salt, natural flavors, soy lecithin, and cinnamon.

In another embodiment, the food product is a jelly bean. In another embodiment, the jelly bean contains the active agents and jelly bean ingredients, including but not limited to cane sugar, tapioca syrup, citric acid, apple juice from concentrate, lime juice from concentrate, raspberry juice from puree, pear juice from concentrate, natural flavor, thiamine hydrochloride, riboflavin, niacinamide, ascorbic acid, potassium citrate, sodium citrate, sodium lactate, confectioners glaze, vegetable and fruit juice for coloring, curcumin, beeswax, carnauba wax, and salt.

In another embodiment, the food product is a sports gel. In another embodiment, the sports gel contains the active agents and sports gel ingredients, including but not limited to maltodextrin, water, fructose, leucine, sea salt, citric acid, natural flavor, potassium citrate, sodium citrate, calcium carbonate, valine, green tea (leaf) extract, gellan gum, isoleucine, sunflower oil, sodium benzoate, and potassium sorbate.

In another embodiment, the food product is a gummy. In another embodiment, the gummy contains the active agents and gummy ingredients, including but not limited to cane sugar, glucose, water, citrus pectin, citric acid, trisodium citrate, natural flavor (strawberry), and natural color (black carrot concentrate).

In another embodiment, the food product is a sports chew. In another embodiment, the sports chew contains the active agents and sports chew ingredients, including but not limited to brown rice syrup, evaporated cane juice, brown rice syrup solids, pectin, citric acid, colored with black carrot juice concentrate, natural flavor, sunflower oil, and carnauba wax.

The dosage of each compound of the claimed combinations depends on several factors, including: the administration method, the condition to be treated, the severity of the condition, whether the condition is to be treated or prevented, and the age, weight, and health of the person to be treated. Additionally, pharmacogenomic (the effect of genotype on the pharmacokinetic, pharmacodynamic or efficacy profile of a therapeutic) information about a particular patient may affect the dosage used.

It is to be understood that in the methods described herein, the individual components of a co-administration, or combination can be administered by any suitable means, contemporaneously, simultaneously, sequentially, separately or in a single pharmaceutical formulation. Where the co-administered compounds or compositions are administered in separate dosage forms, the number of dosages administered per day for each compound may be the same or different. The compounds or compositions may be administered via the same or different routes of administration. The compounds or compositions may be administered according to simultaneous or alternating regimens, at the same or different times during the course of the therapy, concurrently in divided or single forms.

In another embodiment, the composition is administered twice daily. In another embodiment, the composition is administered three times daily. In another embodiment, the composition is administered in the morning and evening. In another embodiment, the composition is administered once a week. In another embodiment, the composition is administered once a month. In another embodiment, the composition is administered to the subject for at least 3 months. In another embodiment, the composition is administered to the subject for at least 4 months, at least 6 months, at least 8 months, at least 9 months, at least 10 months, at least 12 months, at least 14 months, at least 16 months, or at least 18 months.

In another embodiment, the subject is a male. In another embodiment, the subject is a female.

In addition to the illustrative dosages and dosing protocols described herein, it is to be understood that an effective amount of any one or a mixture of the compounds described herein can be readily determined by the attending diagnostician or physician by the use of known techniques and/or by observing results obtained under analogous circumstances. In determining the effective amount or dose, a number of factors are considered by the attending diagnostician or physician, including, but not limited to the species of mammal, including human, its size, age, and general health, the specific disease or disorder involved, the degree of or involvement or the severity of the disease or disorder, the response of the individual patient, the particular compound administered, the mode of administration, the bioavailability characteristics of the preparation administered, the dose regimen selected, the use of concomitant medication, and other relevant circumstances.

It is to be understood that in every instance disclosed herein, the recitation of a range of integers for any variable describes the recited range, every individual member in the range, and every possible subrange for that variable. For example, the recitation that n is an integer from 0 to 8, describes that range, the individual and selectable values of 0, 1, 2, 3, 4, 5, 6, 7, and 8, such as n is 0, or n is 1, or n is 2, etc. In addition, the recitation that n is an integer from 0 to 8 also describes each and every subrange, each of which may for the basis of a further embodiment, such as n is an integer from 1 to 8, from 1 to 7, from 1 to 6, from 2 to 8, from 2 to 7, from 1 to 3, from 2 to 4, etc.

It is also to be understood that unless otherwise indicated the recitation of a numerical value necessarily reflects the relative precision of the numerical value. For example, the recitation of a number with a specified precision based on significant figures necessarily includes a range of values that would match that number after appropriate rounding. For example, the recitation of the number 1 with a single significant figure is understood to properly refer to a range of values from 0.5 to 1.4. Similarly, the recitation of the number 1.0 with two significant figures is understood to properly refer to a range of values from 0.95 to 1.04. The relative precision of the numerical value can be further indicated by modifying with the term “about” to indicate that the modified number has lower precision.

As used herein, the term “composition” generally refers to any product comprising the specified ingredients in the specified amounts, as well as any product which results, directly or indirectly, from combinations of the specified ingredients in the specified amounts. It is appreciated that certain functional groups, such as the hydroxy, amino, and like groups form complexes and/or coordination compounds with water and/or various solvents, in the various physical forms of the compounds. It is to be understood that the compositions described herein may be prepared from isolated compounds described herein or from salts, solutions, hydrates, solvates, and other forms of the compounds described herein. It is also to be understood that the compositions may be prepared from various amorphous, non-amorphous, partially crystalline, crystalline, and/or other morphological forms of the compounds described herein. It is also to be understood that the compositions may be prepared from various hydrates and/or solvates of the compounds described herein. In addition, it is to be understood that the compositions may be prepared from various co-crystals of the compounds described herein. Accordingly, such pharmaceutical compositions that recite compounds described herein are to be understood to include each of, or any combination of, the various morphological forms and/or solvate or hydrate forms of the compounds described herein.

Illustratively, compositions may include one or more carriers, diluents, and/or excipients. The compounds described herein, or compositions containing them, may be formulated in a therapeutically effective amount in any conventional dosage forms appropriate for the methods described herein. The compounds described herein, or compositions containing them, including such formulations, may be administered by a wide variety of conventional routes for the methods described herein, and in a wide variety of dosage formats, utilizing known procedures (see generally, Remington: The Science and Practice of Pharmacy, (21^{st} ed., 2006)).

As used herein, the term “solvates” refers to compounds described herein complexed with a solvent molecule. It is appreciated that compounds described herein may form such complexes with solvents by simply mixing the compounds with a solvent, or dissolving the compounds in a solvent. It is appreciated that where the compounds are to be used as pharmaceuticals, such solvents are pharmaceutically acceptable solvents. It is further appreciated that where the compounds are to be used as pharmaceuticals, the relative amount of solvent that forms the solvate should be less than established guidelines for such pharmaceutical uses, such as less than International Conference on Harmonization (ICH) Guidelines. It is to be understood that the solvates may be isolated from excess solvent by evaporation, precipitation, and/or crystallization. In another embodiment, the solvates are amorphous, and in other embodiments, the solvates are crystalline.

In each of the foregoing and each of the following embodiments, unless otherwise indicated, it is also to be understood that the formulae include and represent any and all crystalline forms, partially crystalline forms, and non-crystalline and/or amorphous forms of the compounds, including partially ordered forms, disordered forms, liquid crystal forms, and meso phases of any of the foregoing.

In each of the foregoing and each of the following embodiments, unless otherwise indicated, it is also to be understood that the formulae include and represent each possible isomer, such as stereoisomers and geometric isomers, both individually and in any and all possible mixtures.

As used herein, the term “inhibiting” generally includes its generally accepted meaning which includes prohibiting, preventing, restraining, slowing, stopping, and/or reversing progression, severity of the disease and/or any resultant symptom of the disease. As such, the methods described herein include both clinical therapeutic and/or prophylactic administration, as appropriate.

The term “therapeutically effective amount” as used herein, refers to that amount of active compound or pharmaceutical agent that elicits the biological or medicinal response in a tissue system, animal or human that is being sought by a researcher, veterinarian, medical doctor or other clinician, which includes alleviation of the symptoms of the disease or disorder being treated. In one aspect, the therapeutically effective amount is that which may treat or alleviate the disease or symptoms of the disease at a reasonable benefit/risk ratio applicable to any medical treatment. However, it is to be understood that the total daily usage of the compounds and compositions described herein may be decided by the attending physician within the scope of sound medical judgment. The specific therapeutically effective dose level for any particular patient will depend upon a variety of factors, including the disorder being treated and the severity of the disorder; activity of the specific compound employed; the specific composition employed; the age, body weight, general health, gender, and diet of the patient: the time of administration, route of administration, and rate of excretion of the specific compound employed; the duration of the treatment; drugs used in combination or coincidentally with the specific compound employed; and like factors well known to the researcher, veterinarian, medical doctor or other clinician of ordinary skill.

It is also appreciated that the therapeutically effective amount, whether referring to monotherapy or combination therapy, is advantageously selected with reference to any toxicity, or other undesirable side effect, that might occur during administration of one or more of the compounds described herein. Further, it is appreciated that the co-therapies described herein may allow for the administration of lower doses of compounds that show such toxicity, or other undesirable side effect, where those lower doses are below thresholds of toxicity or lower in the therapeutic window than would otherwise be administered in the absence of a cotherapy.

The term “administering” as used herein includes all means of introducing the compounds and compositions described herein to the host animal, including, but are not limited to, oral (po), intravenous (iv), intramuscular (im), subcutaneous (sc), transdermal, inhalation, buccal, ocular, sublingual, vaginal, rectal, and the like. The compounds and compositions described herein may be administered in unit dosage forms and/or formulations containing conventional nontoxic pharmaceutically acceptable carriers, adjuvants, and/or vehicles.

As used herein, the term “carrier” generally refers to any ingredient other than the active components in a formulation. The choice of carrier will to a large extent depend on factors such as the particular mode of administration, the effect of the carrier on solubility and stability, and the nature of the dosage form.

In further embodiments, the active agents or compositions thereof are administered one, two, three, or four times daily. In another embodiment, the composition is administered once daily. In another embodiment, the composition is administered twice daily. In another embodiment, the composition is administered three times daily. In another embodiment, the composition is administered four times daily. In another embodiment, the active agents or compositions thereof are administered in the morning and evening. In another embodiment, the active agents or compositions thereof are administered one, two, three, or four times weekly. In another embodiment, the active agents or compositions thereof are administered one, two, three, or four times monthly.

In certain embodiments, the term “prevent” or “preventing” as related to a disease or disorder may refer to a compound that, in a statistical sample, reduces the occurrence of the disorder or condition in the treated sample relative to an untreated control sample, or delays the onset or reduces the severity of one or more symptoms of the disorder or condition relative to the untreated control sample.

The terms “treat,” “treating” or “treatment,” as used herein, may include alleviating, abating or ameliorating a disease or condition symptoms, ameliorating the underlying causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition either prophylactically and/or therapeutically.

In certain embodiments, the term “delay” or “delaying” as related to a disease or disorder may refer to a compound that, in a statistical sample, delays or postpones the occurrence of the disorder or condition in the treated sample relative to an untreated control sample, or delays the onset or reduces the severity of one or more symptoms of the disorder or condition relative to the untreated control sample.

As used herein, “subject” may refer to a mammal. In another embodiment, the mammal is a human. In another embodiment, the mammal is a dog. In another embodiment, the mammal is livestock. In another embodiment, the livestock is selected from the group consisting of cattle, sheep, goats, swine, poultry, and equine animals. In another embodiment, the subject is a male. In another embodiment, the subject is a female. In another embodiment, the human is at least 18 years, at least 20 years, at least 25 years, at least 30 years, at least 35 years, at least 40 years, at least 45 years, at least 50 years, at least 55 years, at least 60 years, at least 65 years, at least 70 years, at least 75 years, or at least 80 years of age.

As used herein, “α-ketoglutarate” or AKG comprises derivatives of α-ketoglutarate (e.g., the derivatives set forth in MacKenzie, et al. (2007) Mol Cell Biol 27(9):3282-3289)), analogues of α-ketoglutarate (e.g., phosphonate analogues (e.g., those recited in Bunik, et al. (2005) Biochemistry 44(31):10552-61), esters of α-ketoglutarate (e.g., dimethyl α-ketoglutarate and octyl α-ketoglutarate), and various species specific analogues, e.g., human α-ketoglutarate, porcine α-ketoglutarate, murine α-ketoglutarate, bovine α-ketoglutarate, and the like.

In another embodiment, the compositions disclosed herein comprise AKG. Alpha-ketoglutarate or α-ketoglutarate (Formula 1) is also known as 2-oxopentanedioic acid, 2-ketoglutaric acid, 2-oxoglutaric acid, and oxoglutaric acid. At physiological pH, α-ketoglutarate exists in one or more deprotonated forms, such as those depicted as Formulae 2. A-ketoglutarate is an intermediate in the Krebs cycle of eukaryotic organisms and is biosynthesized from isocitrate (in the Krebs cycle process) or L-glutamate (via alanine transaminase) in such organisms. Both α-ketoglutarate and its corresponding salts are commercially available, either via preparation from fermentation cultures (for example see U.S. Pat. No. 2,776,926) or chemical synthesis from closely related compounds.

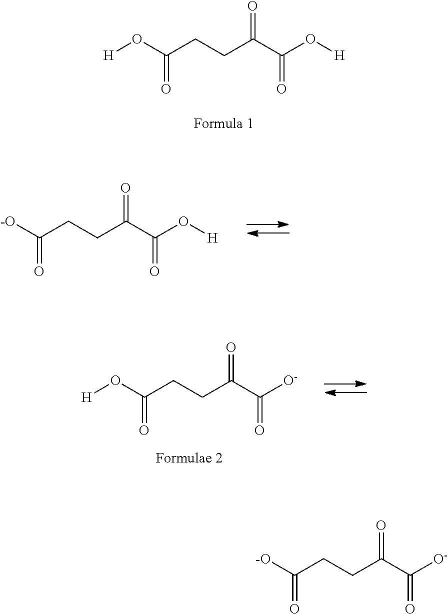

Consistent with its role in energy generation via the Krebs cycle, α-ketoglutarate is an important regulator of bioenergetics in cells and is implicated as an inhibitor of ATP synthase subunit β and an indirect inhibitor of the kinase mTOR, a consequence of partial inhibition of the mitochondrial electron transport chain.

In another embodiment, α-ketoglutarate is provided as the free acid (α-ketoglutaric acid). In another embodiment, α-ketoglutarate is provided as a mono salt or bis salt. In other embodiments, α-ketoglutarate is provided as a monosodium salt, a disodium salt, a monopotassium salt, or a dipotassium salt. In yet further embodiments, α-ketoglutarate is provided as a mono- or di-valent salt with other cations described in the U.S. FDA Orange Book. Such cations include calcium, diolamine, lithium, lysine, magnesium, meglumine, olamine, tromethamine, and zinc. In further embodiments, salts of α-ketoglutarate are provided as anhydrous salts, hemihydrates, monohydrates, or dihydrates.

Further disclosed herein, in certain aspects, are compositions that comprise α-ketoglutarate salt. In another embodiment, α-ketoglutarate is provided as a calcium salt (Ca-AKG). In another embodiment, calcium α-ketoglutarate can be a hydrate calcium α-ketoglutarate. In another embodiment, calcium α-ketoglutarate can be a mono-hydrate calcium α-ketoglutarate. In another embodiment, calcium α-ketoglutarate can be hemi-hydrate calcium α-ketoglutarate. In another embodiment, calcium α-ketoglutarate can be anhydrous calcium α-ketoglutarate.

In another embodiment, the compositions disclosed herein comprise an ester of α-ketoglutarate. In another embodiment, the ester of α-ketoglutarate is a methyl ester of α-ketoglutarate. In another embodiment, the ester of α-ketoglutarate is a dimethyl ester of α-ketoglutarate. In another embodiment, the ester of α-ketoglutarate is an ethyl ester of α-ketoglutarate. In another embodiment, the ester of α-ketoglutarate is a diethyl ester of α-ketoglutarate.

In another embodiment, AKG or Ca-AKG is combined with fish oil. In another embodiment, AKG or Ca-AKG is formulated with essential amino acids, including one or more of L-histidine, L-isoleucine, L-leucine, L-lysine, L-methionine, L-phenylalanine, L-threonine, or L-valine. In another embodiment, CaAKG or AKG is combined with nordihydroguaiaretic acid.

Methylenetetrahydrofolate reductase (MTHFR) is an enzyme that regulates folate metabolism in the body. Specifically, MTHFR converts 5,10-methylenetetrahydrofolate into 5-methyltetrahydrofolate (5-MTHF), the active form of folate. 5-MTHF plays a key role in single carbon transfer (methylation) reactions involved in the synthesis of nucleotides for DNA and RNA production; manufacture of S-adenosylmethionine (SAM-e); methylation of DNA, proteins, neurotransmitters, and phospholipids; and remethylation of homocysteine to methionine. MTHFR is important in converting synthetic folic acid commonly ingested as a vitamin supplement. Synthetic folic acid lacks coenzyme activity, and must be converted to active 5-MTHF.

Mutations within the MTHFR gene may significantly reduce its activity. These mutations often occur as single nucleotide polymorphisms (SNPs). Subjects acquiring MTHFR SNPs from both parents (A/A, homozygous positive) may have significantly reduced (by 80%) MTHFR activity and a marked deficiency of active 5-MTHF. Subjects receiving a SNP from only one parent (A/G, heterozygous positive) may have suboptimal (approximately 40% reduced) MTHFR function. Three well-understood MTHFR SNPs are 677C to T (C667T also called rs1801133), 1298A to C (A1298C, also called rs1801131), and rs1999594. Genetic testing for the presence of these SNPs can help assess how well homocysteine can be cleared from the blood in affected subjects.

Folic acid and folate (also referred to as vitamin B9) help the body make new red blood cells. Red blood cells carry oxygen to all the parts of your body, and a deficiency of red blood cells can cause anemia. Anemia occurs when blood cannot carry enough oxygen to the body, and can make the subject pale, tired, or weak. Anemia includes folate-deficiency anemia. It is appreciated that sufficient folic acid and folates are especially important for health in women. Folic acid and folates protect unborn children against serious birth defects, including neural tube defects. These birth defects happen in the first few weeks of pregnancy, often before a woman knows she is pregnant. Folic acid and folates may also help prevent other types of birth defects and early pregnancy loss (miscarriage). Reportedly, as many as 40% of humans suffer one or more folate metabolism deficiencies that may lead to the accumulation of by-products, such as homocysteine, from an inefficient folate cycle.

Homocysteine is an amino acid synthesized by the body through the demethylation of methionine. In the presence of adequate B vitamins, homocysteine is either irreversibly degraded to cysteine or it is re-methylated back to methionine, an essential amino acid. An elevated homocysteine level is reported to be an independent risk factor for ischemic stroke, thrombotic, and cardiovascular diseases. Folates, vitamin B6, and vitamin B12 are all necessary for the proper conversion of homocysteine into methionine. A deficiency in any of these vitamins can cause homocysteine levels to rise. Though homocysteine is a necessary amino acid in methylation cycles, at elevated levels, it can become toxic to cells, damage blood vessels, promote oxidation, elicit an autoimmune response, increase the risk of atherosclerosis, and/or contribute to dementia, depression, and neural tube defects.

Though homocysteine is generated from methionine, it is continuously recycled back to methionine (or on to cystathionine) in order to avoid accumulation. A number of genes play an active role in the overall processing of homocysteine and associated SNPs can disrupt that process and lead to toxic homocysteine levels. Methionine/homocysteine balance is important for optimal health, including proper methylation by donating methyl groups for DNA methylation, including gene regulation, regulation of neurotransmitters, such as epinephrine, norepinephrine, and dopamine, detoxification of catecholamines obtained from the environment; and drug clearance, including phase II liver detoxification.

Homocysteine is also a precursor in the biosynthesis of L-cysteine for glutathione, which is important for the detoxification of electrophilic compounds, such as metals. Elevated serum homocysteine is a widely accepted marker for methionine/homocysteine imbalance, which is a genetically controlled process. Elevated homocysteine levels can lead to accelerated aging, cardiovascular disease, and neurodegenerative disorders.

Alkaline Phosphatase (ALPL) is an enzyme responsible for the growth and development of bones and teeth. It also plays an important role in removing excess vitamin B6 from the blood. The human body cannot produce vitamin B6, and daily requirements of vitamin B6 are obtained from food or vitamin supplementation. When the body is unable to get the required amount of vitamin B6, it leads to a deficiency. There are two types of vitamin B6 deficiencies. Primary vitamin B6 deficiency results from the reduced or inadequate intake through food. Secondary vitamin B6 deficiency can be caused by malabsorption, alcoholism, hemolysis, protein-energy malnutrition, and the use of pyridoxine inactivating drugs such as penicillamine, corticosteroids, anticonvulsants. Vitamin B6 deficiency is generally rare.

An excess of vitamin B6 can also be dangerous as it can cause progressive and severe sensory neuropathy, which is primarily characterized by ataxia (impaired body coordination). The severity of the symptoms is dependent on the level of the excess. Excessive vitamin B6 consumption can also cause painful dermatological lesions, photosensitivity, and GI symptoms such as acid reflux and nausea.

Several genetic mutations have been identified in the ALPL gene that might interfere in vitamin B6 clearance. Subjects acquiring SNPs from both parents (C/C, homozygous positive) generally have significantly increased ALPL activity, and average vitamin B6 blood levels significantly lower than normal. Subjects receiving a SNP from only one parent (C/T, heterozygous positive) generally have suboptimal ALPL function and have average vitamin B6 blood levels slightly lower than normal. An illustrative ALPL SNP is rs4654748. Genetic testing for the presence of these SNPs can help assess how well vitamin B6 is cleared from the blood in affected subjects. ALPL SNPs may require that subjects supplement vitamin B6 intake than the recommended daily requirements of 1.3 μg, include foods rich in Vitamin B6, and/or supplement with Active B complex of Pyridoxal-5′-phosphate (B6) and Riboflavin-5′-phosphate (B2).

Fucosyltransferase (FUT2) is an enzyme that provides the ability to produce glycans (fiber-like molecules) found in saliva and in the mucus of our intestine. In saliva and mucus, the glycans are part of the body's natural defense system, and may include a prebiotic known as 2′-fucosyllactose (2′FL) which has wide-ranging beneficial effects in the gut, brain, and overall health. It has been reported that 20% of the population produces α1,2-fucosyltransferase, FUT2 G/G (rs602662), that mediates the fucosylation of oligosaccharides to form H type 1 and H type 2 antigens. Those H antigens mediate the adhesion of various gastric pathogens, such as Helicobacter pylori to the gastric and duodenal mucosa. Overgrowth of gastric bacteria, such as Helicobacter pylori, has been associated with vitamin B12 deficiency. The reduced activity of the FUT2 enzyme with the A allele may decrease susceptibility to bacterial infection and indirectly lower the risk of vitamin B12 malabsorption, thereby resulting in higher vitamin B12 concentrations in A allele carriers. Vitamin B12 is an essential co-factor in the folate metabolism pathway, and a vitamin B12 deficiency can lead to high levels of homocysteine and high levels of unmethylated DNA.

Subjects with FUT2 A/G or G/G SNPs are present generally need to supplement vitamin B12, such as methylcobalamin, above the recommended daily requirements and/or include foods rich in vitamin B12. It is understood herein that using the active form of vitamin B12 helps ensure that the subject has the necessary methyl groups to regenerate the active folate.

In another embodiment, the subject is pre-screened for a DNA methylation profile. In another embodiment, the DNA methylation profile of the subject is comparable to a DNA methylation profile of a control subject. In another embodiment, the control subject is a subject administered a placebo. In another embodiment, the control subject is at least of same age as the subject. In another embodiment, the composition alters the DNA methylation profile of the subject. In another embodiment, the altered DNA methylation profile of the subject is comparable to a second control subject who is at least 2 years younger than the subject. In another embodiment, the altered DNA methylation profile of the subject is comparable to a second control subject who is at least 3 years younger than the subject. In another embodiment, the altered DNA methylation profile of the subject is comparable to a second control subject who is at least 4 years younger than the subject. In another embodiment, the altered DNA methylation profile of the subject is comparable to a second control subject who is at least 5 years younger than the subject. In another embodiment, the altered DNA methylation profile of the subject is comparable to a second control subject who is at least 6 years younger than the subject. In another embodiment, the altered DNA methylation profile of the subject is comparable to a second control subject who is at least 7 years younger than the subject. In another embodiment, the altered DNA methylation profile of the subject is comparable to a second control subject who is at least 8 years younger than the subject. In another embodiment, the altered DNA methylation profile of the subject is comparable to a second control subject who is at least 9 years younger than the subject. In another embodiment, the altered DNA methylation profile of the subject is comparable to a second control subject who is at least 10 years younger than the subject.

In another embodiment, the subject is screened for mutations in MTHFR, ALPL, and/or FUT2.

In another embodiment, the disclosure provides methods for treating, delaying onset, or delaying progression of frailty using the active agents or compositions thereof described herein. The term “frailty” refers to a biological syndrome of decreased reserve and resistance to stressors due to decline in multiple physiological systems. Subjects suffering from frailty have improved likelihood of adverse health outcomes to events that stress one or more of their physiological systems. In humans, frailty frequently presents via non-specific symptoms, falls, delirium, fluctuating disability, or a combination thereof. Non-specific symptoms include extreme fatigue, unexplained weight loss, and frequent infections. Falls include hot falls (minor illness reducing postural balance below a threshold to maintain stability) or spontaneous falls (vital postural systems declining as a result of declines in vision, balance, and strength). Delirium refers to rapid onset of fluctuating confusion and impaired awareness. Fluctuating disability refers to day-to-day instability in the ability of a patient to function independently.

Various clinical scoring and evaluation systems for frailty are known and are suitable for assessing the effects of treatment on frailty. Illustratively, frailty is evaluated in humans using the 70-item CSHA Frailty Index (see, for e.g. Theou et al. Age Ageing 42: 614-619 (2013)).

For example, clinical scoring and evaluation systems for frailty include the presence and/or severity of current diseases, ability in daily living and physical signs from the clinical and neurological examinations (see items in Table 1 below). Each deficit is dichotomized or trichotomized and mapped to the interval 0-1 (i.e. individual items had scores of 0, 0.33, 0.50, 0.67 or 1.0), representing the occurrence and severity of the problem. For each person, a 70-dimentional vector is constructed, such that a person with 5 deficits would have a score of 5/70=0.071.

TABLE 1
List of measures used to construct Frailty Index

| | | Limitations with |
|---|---|---|
| Self-rated health | Long-term illness | Activities |
| In hospital last 12 | Heart attack | High blood pressure |
| months | | |
| High blood | Stroke or cerebral | Diabetes or high blood |
| cholesterol | vascular Disease | sugar |
| Chronic lung disease | Asthma | Arthritis |
| Osteoporosis | Cancer | Stomach or duodenal |
| | | ulcer |
| Parkinson disease | Cataracts | Hip or femoral |
| | | fracture |
| Pain in any joint | Heart trouble or | Breathlessness |
| | angina | |
| Persistent cough | Swollen legs | Sleeping problems |
| Falling down | Fear of falling down | Dizziness |
| Stomach or intestine | Incontinence | Dentures |
| Problems | | |
| Biting on hard foods | Eyesight | Hearing |
| Walking 100 meters | Sitting for about two | Getting up from a |
| | hours | chair after prolonged |
| | | sitting |
| Climbing several | Stooping, kneeling, or | Reaching or extending |
| flights of Stairs | Crouching | arm above shoulder |
| | | level |
| Pulling or pushing | Lifting or carrying | Picking up a small |
| large Objects | weights over 10 | coin from a table |
| | pounds/5 kilos | |
| Dressing | Walking across a | Bathing or showering |
| | room | |
| Eating | Getting in or out of | Using the toilet |
| | bed | |
| Using a map to figure | Preparing a hot meal | Shopping for groceries |
| out how to get around | | |
| Making telephone | Taking medications | Doing work around |
| calls | | the house or garden |
| Managing money | Vigorous activities | Moderate Activities |
| Orientation | Mathematical | Verbal fluency score |
| | performance | |
| Delayed recall test | Depression | Pessimism |
| Suicidality | Sleep | Interest |
| Appetite | Fatigue | Concentration |

Further detail on application of the CSHA frailty index (e.g. calculation of scores for individual measures) can be found in other publications in the field, for e.g. in Searle et al. A standard procedure for creating a frailty index. BMC Geriatrics 8:24 (2008). An example of the use of the CSHA frailty index in humans can be found in the Examples, where selection of pre-frail individuals for treatment and evaluation of pharmacological treatment of frailty is described.

In another embodiment, frailty is evaluated in non-human animals, such as mice. Recognized signs of frailty in mice correspond to many of those in humans, and involve metabolic (e.g. body temperature, body weight), integumental (e.g. alopecia, loss of fur color, dermatitis, loss of whiskers, grooming), physical/musculoskeletal (e.g. tumors, distended abdomen, kyphosis, tail stiffening, gait disorder, tremor, decreased forelimb grip strength, body condition/muscle wasting/obesity), vestibulocochlear/auditory (e.g. vestibular disturbance, hearing loss), ocular/nasal (e.g. cataracts, corneal opacity, eye discharge, microphthalmia, vision loss, increased menace reflex, nasal discharge), digestive/urogenital (e.g. malocclusions, rectal prolapse, vaginal/uterine/penile prolapse, diarrhea), respiratory (e.g. abnormal breathing rate or depth), and discomfort symptoms (e.g. increased mouse grimace scale, piloerection).

In another embodiment, frailty is assessed in mice via a 31-item clinical frailty index encompassing the 31 example phenotypes recited above as described in, for e.g. Whitehead et al. J Gerontol A Biol Sci Med Sci 69:621-632 (2014). Clinical examinations are performed at approximately the same time every 2 to 3 months, and involve body weight and surface temperature measurement by abdominal infrared, followed by a clinical exam to evaluate the 31 frailty phenotypes. The severity of each deficit is rated on a scale, with 0 given for no sign of a deficit, 0.5 for a mild deficit, and 1 for a severe deficit. Deficits in body weight (g) and body surface temperature (° C.) are scored in quantiles between 0 and 1 based on number of standard deviations from reference values in young adult animals (0.25, 0.5, 0.75, and 1.0) according to how many standard deviations the score varies from the mean (1 is >3SD). The sum of the scores for each parameter produce the final 31-item frailty index, which can be compared between individual mice according to standard statistical techniques to assess frailty.

In another embodiment, frailty is assessed in mice via an abbreviated with an eight-item functional frailty index as described in, for e.g. Whitehead et al. J Gerontol A Biol Sci Med Sci 69:621-632 (2014) and Parks et al. J Gerontol A Biol Sci Med Sci. 67:217-227 (2012). In this method, 7 performance parameters based on open-field behavior of mouse subjects are assessed: 1) total distance moved in 10 minutes; 2) maximal distance moved between bouts of inactivity; 3) total duration of movement (seconds); 4) percent of total time spent moving; 5) the change in direction per unit distance moved, called meander (degrees/cm; from 0° to 180°); 6) the average velocity of movement over 10 minutes (cm/s); and 7) rearing frequency (number of occurrences/10 min). An eighth non-movement parameter, weight, is additionally assessed. Open-field assessments are performed between 10 am and noon each day. Mice are weighed and activity was recorded with automated video tracking software for 10 minutes in an open-field arena. Videos are digitized with an analog-to-digital converter and analyzed with video tracking analysis software to obtain values for the parameters used to create the eight-item frailty index. Mean and standard deviation for each of these parameters are calculated and assigned to a score quantile between 0 and 1 (0.25, 0.5, 0.75, and 1.0) according to how many standard deviations the score varies from the mean (1 is >3SD). The parameters are added, and divided by eight to receive a frailty index score between 0 and 1 for the mouse subjects. Higher scores correspond to increasingly frail mice.

In another embodiment, the disclosure provides methods of helping to maintain health using the active agents or compositions thereof described herein. In another embodiment, helping to maintain health comprises helping to maintain a healthy metabolism. In another embodiment, helping to maintain a healthy metabolism comprises helping to maintain a healthy body temperature. In another embodiment, helping to maintain a healthy metabolism comprises helping to maintain a healthy body weight.

In another embodiment, helping to maintain health comprises helping to maintain a healthy musculoskeletal system. In another embodiment, helping to maintain a healthy musculoskeletal system comprises helping to maintain grip strength. In another embodiment, helping to maintain a healthy musculoskeletal system comprises helping to maintain normal spine curvature. In another embodiment, helping to maintain a healthy musculoskeletal system comprises helping to maintain normal gait. In another embodiment, helping to maintain a healthy musculoskeletal system comprises helping to maintain normal muscle mass. In another embodiment, helping to maintain a healthy musculoskeletal system comprises helping to maintain and/or improve balance. In another embodiment, helping to maintain a healthy musculoskeletal system comprises helping to maintain and/or improve locomotion.

In another embodiment, helping to maintain health comprises helping to maintain a healthy auditory system. In another embodiment, helping to maintain health comprises helping to maintain a healthy ocular system. In another embodiment, helping to maintain health comprises helping to maintain normal vision. In another embodiment, helping to maintain health comprises helping to maintain a healthy digestive system. In another embodiment, helping to maintain health comprises helping to maintain a healthy urogenital tract. In another embodiment, helping to maintain health comprises helping to maintain a healthy respiratory system.

In another embodiment, helping to maintain health comprises helping to maintain a healthy cardiovascular system. In another embodiment, helping to maintain health comprises helping to maintain a healthy body weight.

In another embodiment, helping to maintain health comprises helping to maintain a healthy senescence-associated secretory phenotype (SASP). In another embodiment, helping to maintain health comprises helping to maintain a healthy level of secretion of SASP factors. In another embodiment, helping to maintain health comprises helping to maintain a healthy level of secretion of SASP factors, wherein SASP factors comprise an interleukin (IL), a chemokine, an inflammatory factor, a growth factor, a protease, an extracellular matrix component, or a combination thereof. In another embodiment, helping to maintain health comprises helping to maintain healthy level of secretion of SASP factors comprising IL-1β, IL-3, IL-6, IL-7, MIP-1β, TNF-α, CCL2, MMP3, or a combination thereof.

In another embodiment, the disclosure provides methods of treatment for extending healthspan using the active agents or compositions thereof disclosed herein. Healthspan refers to the period of time during which an individual meets one or more selected measures of health. An increase in healthspan refers to an extension in the period of health, according to such measures, as compared to the period of health in a control population. Examples of selected measures of health that are evaluated in a human population to assess healthspan include one or more age-related phenotypes such as energetics/metabolism (e.g. elevated insulin, insulin resistance, elevated fasting blood glucose+GTT, elevated Hb A1c, adiponectin, elevated DEXA/abdominal adiposity, increased IGF-I, decreased T3, elevated low-density lipoprotein, decreased high-density lipoprotein, elevated triglycerides), skeletal muscle function (e.g. decreased hand grip strength, decreased mobility), cardiopulmonary function (e.g. decreased VO2 max, elevated blood pressure, decreased pulse wave velocity, intima media thickness, decreased left ventricular diastolic function, increased left ventricular diastolic pressure), inflammation and immune function (e.g. decreased lymphocyte number, decreased lymphoid/myeloid ratio, elevated CRP, elevated IL-6, elevated TNF-α), sensory function (e.g. decreased visual acuity, decreased nerve conduction velocity), cognition (e.g. decreased score on cognitive function tests like the MMSE/AMTS/GPAC, impaired activity via fMRIs), cellular senescence (e.g. graying hair), and pathology (e.g. renal, cardiac, pulmonary, breast, or prostate tissue evaluation to evaluate age-related tissue hypertrophy or dysplasia).

Examples of non-human animals used for assessment of lifespan-extension or healthspan interventions include C. elegans, D. melanogaster, and M. musculus. Use of D. melanogaster or M. musculus to evaluate lifespan or healthspan interventions are found in, for e.g. Bauer et al. Proc Natl Acad Sci USA. 101:12980-5 (2004) and Selman et al. FASEB J 22:807-18 (2008).

In another embodiment, the disclosure provides methods of compressing morbidity using the active agents or compositions thereof described herein. Compression of morbidity occurs if the age of first appearance of aging manifestations and chronic disease symptoms increases more rapidly than life expectancy. The period between marker of morbidity (e.g. first heart attack, first dyspnea from emphysema, first disability from osteoarthritis, first memory loss of a certain magnitude) and the end of life is shortened when the average onset age of the marker increases more rapidly than life expectancy from the same age. This disproportionally increases the healthy years of life, and dramatically reduces the end stage costs of healthcare.

In another embodiment, the disclosure provides methods of altering senescence-associated secretory phenotype (SASP) using the active agents or compositions thereof described herein. In another embodiment, the methods disclosed herein alter SASP of a senescent cell in a subject in need thereof. A “senescent cell” is generally thought to be derived from a cell type that typically replicates, but as a result of aging or other event that causes a change in cell state, can no longer replicate. The nucleus of senescent cells is often characterized by senescence-associated heterochromatin foci and DNA segments with chromatin alterations reinforcing senescence. It remains metabolically active and commonly adopts a senescence associated secretory phenotype (SASP). The SASP can include a combination of inflammatory cytokines, growth factors, and proteases. Without being bound by a particulary theory, the SASP secreted by senescent cells can contribute to the onset and progression of age-related diseases.

In another embodiment, the disclosure provides methods of maintaining hair density using the active agents or compositions thereof described herein. In another embodiment, hair density is maintained in a subject that has alopecia. In another embodiment, maintaining hair density comprises maintaining a healthy scalp. In another embodiment, maintaining hair density comprises maintaining healthy hair follicles. In another embodiment, maintaining hair density comprises maintaining healthy hair shafts. In another embodiment, maintaining hair density comprises maintaining healthy hair bulbs.

In another embodiment, the disclosure provides methods of maintaining hair pigmentation using the active agents or compositions thereof described herein. In another embodiment, maintaining hair pigmentation comprises maintaining a normal level of melanin. In another embodiment, hair pigmentation is maintained in a subject that has low vitamin B12 levels. In another embodiment, maintaining hair pigmentation comprises maintaining a normal level of melanocyte stem cells.

In another embodiment, the disclosure provides methods of re-growing hair in a subject in need thereof using the active agents or compositions thereof described herein. In another embodiment, the subject has alopecia. In another embodiment, the subject has male or female pattern baldness. In another embodiment, the subject has alopecia areata. In another embodiment, the subject has telogen effluvium. In another embodiment, the subject has anagen effluvium. In another embodiment, the subject has alopecia totalis. In another embodiment, the subject has alopecia universalis. In another embodiment, the subject has alopecia barbae. In another embodiment, the subject has alopecia mucinosa. In another embodiment, the subject has traction alopecia. In another embodiment, the subject has scarring alopecia. In another embodiment, the subject has trichotillomania.

In addition, it is to be understood that therapeutically effect doses administered in animal models may be used to calculate corresponding therapeutically effect doses for administration to other host animals, including humans. Illustrative corresponding doses may be calculated using the “Guidance for Industry Estimating the Maximum Safe Starting Dose in Initial Clinical Trials for Therapeutics in Adult Healthy Volunteers” published by FDA, and found at https://www.fda.gov/media/72309/download, and which is incorporated herein in its entirety by reference.

The following examples further illustrate specific embodiments of the invention; however, the following illustrative examples should not be interpreted in any way to limit the invention.

Each publication cited herein is incorporated herein by reference:

### Examples

FORMULATION EXAMPLE. A tablet comprising calcium AKG or calcium AKG monohydrate (500 mg), and one or more of pyridoxine (25 mg), cyanocobalamin (250 mcg), folic acid (300 mcg), and riboflavin (5 mg).

FORMULATION EXAMPLE. A tablet comprising calcium AKG or calcium AKG monohydrate (500 mg) and vitamin A (1500 IU), and one or more of pyridoxine (25 mg), cyanocobalamin (250 mcg), folic acid (300 mcg), and riboflavin (5 mg).

FORMULATION EXAMPLE. A tablet comprising (a) calcium AKG or calcium AKG monohydrate (500 mg), (b) one or more of pyridoxine (25 mg), cyanocobalamin (250 mcg), folic acid (300 mcg), and riboflavin (5 mg), and (c) one or more of vegetable waxes (rice bran wax, carnauba wax, or a combination thereof), stearic acid, and magnesium stearate.

FORMULATION EXAMPLE. A tablet comprising (a) calcium AKG or calcium AKG monohydrate (500 mg) and vitamin A (1500 IU), (b) one or more of pyridoxine (25 mg), cyanocobalamin (250 mcg), folic acid (300 mcg), and riboflavin (5 mg), and (c) one or more of vegetable waxes (rice bran wax, carnauba wax, or a combination thereof), stearic acid, and magnesium stearate.

FORMULATION EXAMPLE. A tablet comprising calcium AKG or calcium AKG monohydrate (500 mg) and vitamin D (500 IU), and one or more of pyridoxine (25 mg), cyanocobalamin (250 mcg), folic acid (300 mcg), and riboflavin (5 mg).

FORMULATION EXAMPLE. A tablet comprising (a) calcium AKG or calcium AKG monohydrate (500 mg) and vitamin D (500 IU), (b) one or more of pyridoxine (25 mg), cyanocobalamin (250 mcg), folic acid (300 mcg), and riboflavin (5 mg), and (c) one or more of vegetable waxes (rice bran wax, carnauba wax, or a combination thereof), stearic acid, and magnesium stearate.

SUSTAINED RELEASE FORMULATION EXAMPLE 1. A tablet formed from an active composition incorporated into a controlled release matrix. The active composition includes calcium AKG or calcium AKG monohydrate (500 mg), and one or more of pyridoxine (25 mg), cyanocobalamin (250 mcg), folic acid (300 mcg), and riboflavin (5 mg). The controlled release matrix includes one or more HPMC (130-160 mg total), and optionally one or more diluents selected from microcrystalline cellulose (180-200 mg), lactose monohydrate (120-150 mg), and colloidal silicon dioxide (5-15 mg).

SUSTAINED RELEASE FORMULATION EXAMPLE 2. A tablet formed from an active composition incorporated into a controlled release matrix. The active composition includes calcium AKG or calcium AKG monohydrate (500 mg) and vitamin A (1500 IU), and one or more of pyridoxine (25 mg), cyanocobalamin (250 mcg), folic acid (300 mcg), and riboflavin (5 mg). The controlled release matrix includes one or more HPMC (130-160 mg total), and optionally one or more diluents selected from microcrystalline cellulose (180-200 mg), lactose monohydrate (120-150 mg), and colloidal silicon dioxide (5-15 mg).

SUSTAINED RELEASE FORMULATION EXAMPLE 3. A tablet formed from an active composition incorporated into a controlled release matrix. The active composition includes (a) calcium AKG or calcium AKG monohydrate (500 mg), (b) one or more of pyridoxine (25 mg), cyanocobalamin (250 mcg), folic acid (300 mcg), and riboflavin (5 mg), and (c) one or more of vegetable waxes (rice bran wax, carnauba wax, or a combination thereof), stearic acid, and magnesium stearate. The controlled release matrix includes one or more HPMC (130-160 mg total), and optionally one or more diluents selected from microcrystalline cellulose (180-200 mg), lactose monohydrate (120-150 mg), and colloidal silicon dioxide (5-15 mg).

SUSTAINED RELEASE FORMULATION EXAMPLE 4. A tablet formed from an active composition incorporated into a controlled release matrix. The active composition includes (a) calcium AKG or calcium AKG monohydrate (500 mg) and vitamin A (1500 IU), (b) one or more of pyridoxine (25 mg), cyanocobalamin (250 mcg), folic acid (300 mcg), and riboflavin (5 mg), and (c) one or more of vegetable waxes (rice bran wax, carnauba wax, or a combination thereof), stearic acid, and magnesium stearate. The controlled release matrix includes one or more HPMC (130-160 mg total), and optionally one or more diluents selected from microcrystalline cellulose (180-200 mg), lactose monohydrate (120-150 mg), and colloidal silicon dioxide (5-15 mg).

SUSTAINED RELEASE FORMULATION EXAMPLE 5. A tablet formed from an active composition incorporated into a controlled release matrix. The active composition includes calcium AKG or calcium AKG monohydrate (500 mg) and vitamin D (500 IU), and one or more of pyridoxine (25 mg), cyanocobalamin (250 mcg), folic acid (300 mcg), and riboflavin (5 mg). The controlled release matrix includes one or more HPMC (130-160 mg total), and optionally one or more diluents selected from microcrystalline cellulose (180-200 mg), lactose monohydrate (120-150 mg), and colloidal silicon dioxide (5-15 mg).

SUSTAINED RELEASE FORMULATION EXAMPLE 6. A tablet formed from an active composition incorporated into a controlled release matrix. The active composition includes (a) calcium AKG or calcium AKG monohydrate (500 mg) and vitamin D (500 IU), (b) one or more of pyridoxine (25 mg), cyanocobalamin (250 mcg), folic acid (300 mcg), and riboflavin (5 mg), and (c) one or more of vegetable waxes (rice bran wax, carnauba wax, or a combination thereof), stearic acid, and magnesium stearate. The controlled release matrix includes one or more HPMC (130-160 mg total), and optionally one or more diluents selected from microcrystalline cellulose (180-200 mg), lactose monohydrate (120-150 mg), and colloidal silicon dioxide (5-15 mg).

DISSOLUTION EXAMPLE. Each of the sustained release formulation Examples 1-6 prepared with a mixture of HPMC 68,000-95,000 (50-70 mg) and HPMC 115,000-150,000 (80-100 mg) was tested in a conventional USP 24 (Apparatus 2, Paddle Apparatus) at 100 rpm. A tablet was placed in the Apparatus and immersed in 900 mL 0.1 N HCl, and exhibited the following release profile for the calcium AKG or calcium AKG monohydrate, and included vitamins:

| 1 hour | 3 hours | 6 hours | 9 hours |
|---|---|---|---|
| 25% ± 10% | 50% ± 10% | 75% ± 10% | ≥90% |

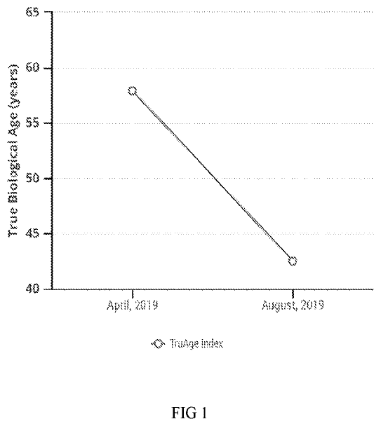

METHOD EXAMPLE. Subject No. 1. A genetic screening of Subject No. 1 showed (a) two copies of the overactive ALPL gene; and (b) FUT2 (A/G and/or G/G) SNPs. ALPL removes vitamin B6 from the blood, and the overactive ALPL gene, suggests a deficiency in vitamin B6 homeostasis. FUT2 mutations are reportedly linked to deficient absorption of vitamin B12. The biological age of Subject No. 1 was determined in a conventional assay performed by Trume Labs, Alameda, CA, and based on DNA methylation. Subject No. 1 was co-administered a composition comprising calcium AKG, 1000 μg (and including retinyl palmitate, 900 mcg and calcium, 190 μg) and a vitamin B complex (vitamin B6, 1.5 μg, as pyridoxal-5-phosphate; folate, 680 mcg DFE, as [6S]-5-Methyltetrahydrofolate, glucosamine salt; vitamin B12, 1000 mcg, as methylcobalamin) once or twice per day for a period of 6 months. The biological age of Subject No. 1 was determined again following the treatment. As shown in FIG. 1, the results showed a net decrease of 15.4±4.2 years in the true biological age of Subject 1. After normalization, the true biological age of Subject 1 following treatment was 6.3 years lower than the chronological age.

METHOD EXAMPLE. Subject No. 2. The biological age of Subject No. 2 was determined in a conventional assay performed by TruMe Labs, Alameda, CA, and based on DNA methylation. Subject No. 2 was co-administered a composition comprising calcium AKG, 1000 μg (and including retinyl palmitate, 900 mcg; and calcium, 190 μg) and a vitamin B complex (riboflavin, 15 μg, as riboflavin-5-phosphate; vitamin B6, 5 μg, as pyridoxal-5-phosphate; folate, 1700 mcg DFE, as [6S]-5-methyltetrahydrofolate, glucosamine salt; vitamin B12, 500 mcg, as methylcobalamin; trimethylglycine (Tp G), 450 μg; N-acetyl-L-cysteine (NAC), 50 μg) once per day for a period of 3 months. The biological age of Subject No. 2 was determined again following the treatment. The results showed a net decrease of 4 years in the biological age of Subject 2, after normalization.

CLINICAL TRIAL EXAMPLE. Exemplary patient experiments or clinical trials include an open label, randomized study to obtain safety and biomarker data on Rejuvant dietary supplement products. The study may have four Groups as outlined in the table below. Groups 1-4 are divided by gender and each Group receives the gender-specific investigational product. Groups 1 and 3 take the commercial serving size of two tablets per day of the gender-specific investigational product. Groups 2 and 4 take three tablets per day of the gender-specific investigational product; these Groups provide data at a larger serving size than the recommended dose. All Groups take the investigational product for six to nine months. The participants are assessed at four time points: Study Initiation, 3-months, 6-months, and 9-months. At each visit a vitals assessment is conducted (height, weight, blood pressure, pulse), blood chemistries, Hemoglobin A1C, C-Reactive Protein, and Uric Acid will be measured. In addition, a blood sample is taken at each visit for metabolite analysis. At visits 1 and 4, a saliva sample is collected for DNA methylation analysis.

The physician exam, blood chemistries, Hemoglobin A1C, C-Reactive Protein, and Uric Acid levels, and the questionnaire allows for an assessment of the safety of the investigational products. These data are used to assess changes in participant metabolism over the course of the study. The blood chemistry and metabolite data are used to calculate the biological age of the participant by one or more published algorithms. DNA methylation analysis of saliva determines the degree of DNA methylation (the cytosine of the CpG dinucleotide is methylated). The degree of DNA methylation is correlated with aging and is used as another measure of the biological age of the participant.

### Inclusion Criteria:

Men 45-65 years
Female 45-65 years

### Exclusion Criteria:

Females of child-bearing age
Pregnant females
Lactating females
Diabetics
Congestive Heart Disease
Myocardial Infarction in the previous year
Undergoing treatment for cancer
Morbidly Obese
Patient groups in the clinical trial

| | | # of | | Dose (# of |
|---|---|---|---|---|
| Group | Test Article | Volunteers | Gender | Tablets) |
| 1 | Rejuvant ™ for Men | 20 | Male | 2 |
| 2 | Rejuvant ™ for Men | 20 | Male | 3 |
| 3 | Rejuvant ™ for Women | 20 | Female | 2 |
| 4 | Rejuvant ™ for Women | 20 | Female | 3 |

Primary outcome measures are used to establish the safety of oral administration of the therapeutic composition. Safety and tolerability of single and multiple doses of a composition described herein are measured by adverse events (AEs), physical examinations (PE), vital signs (VS), laboratory safety tests, urinalysis, and 12-lead electrocardiograms (ECG). Outcomes measured during site visits include: height, weight, blood pressure and pulse, complete blood counts, comprehensive metabolic panel, lipid panel, and blood measures such as hemoglobin A1C, c-reactive protein levels, and uric acid.

Complete Blood Count may include: WBC (White Blood Cell Count), Hemoglobin, Hematocrit, Platelet Count/L, MCV (Mean Corpuscular Volume), MCH (Mean Corpuscular Hemoglobin), MCHC (Mean Corpuscular Hemoglobin Concentration), RBC (Red Blood Cell Count), RDW-CV (Red Cell Distribution Width), MPV (Mean Platelet Volume), Neutrophils, Neutrophil %, Lymphocyte Count, Lymphocyte %, Monocyte Count, Monocyte %, Eosinophils, Eosinophil %, Basophils, Basophil %.

Comprehensive Metabolic Panel may include measuring: Sodium, Potassium, Chloride, Carbon Dioxide (CO₂), Urea Nitrogen (BUN), Creatinine, Glucose, Calcium, AST (Aspartate Aminotransferase), ALT (Alanine Aminotransferase), Alk Phos (Alkaline Phosphatase), Bilirubin, Total, Alkaline Phosphatase, Albumin, Protein, Total, Anion Gap, Glomerular Filtration Rate (eGFR), MDRD Estimate.

Lipid Panel may include: Total Cholesterol, LDL (Low Density Lipoprotein) Cholesterol, HDL (High Density Lipoprotein) Cholesterol, Triglyceride,

The secondary outcome is to observe changes in blood chemistries, metabolites, and the amount of DNA methylation by the oral administration of Rejuvant products per dosing described. The secondary outcome measures may include Metabolic Screen (from blood samples), DNA Methylation Assay (from saliva samples).

### Example Questionnaire

How do you feel today?
How would you describe your daily activity?
How would you rate your physical energy level?
How would you rate your cardiovascular endurance level?
How would you describe your ability to focus?
How would you rate your level of memory recall?
How would you describe your quality of sleep?
How would you rate the youthfulness of your hair?
How many minutes per week do you exercise including light exercise?
Is there any other relevant information about the product you'd like to share?

CLINICAL TRIAL EXAMPLE. Exemplary patient experiments or clinical trials include a placebo-controlled, randomized study to obtain safety and biomarker data on Rejuvant dietary supplement products. The study has four Groups as outlined in the table below. Groups 1-4 are divided by gender and each Group receives the gender-specific investigational product or a placebo. Groups 1 and 3 take the commercial serving size of two tablets per day of the gender-specific investigational product. Groups 2 and 4 will take two placebos per day. All Groups take the tablets for six to nine months. The participants are assessed at four time points: Study Initiation, 3-months, 6-months, and 9-months. At each visit a vitals assessment is conducted (height, weight, blood pressure, pulse), blood chemistries, Hemoglobin A1C, C-Reactive Protein, and Uric Acid, will be measured. In addition, a blood sample is taken at each visit for metabolite analysis. At visits 1 and 4, a saliva sample is collected for DNA methylation analysis.

The physician exam, blood chemistries, Hemoglobin A1C, C-Reactive Protein, and Uric Acid levels, and the questionnaire allows for an assessment of the safety of the investigational products. These data are used to assess changes in participant metabolism over the course of the study. The blood chemistry and metabolite data are used to calculate the biological age of the participant by one or more published algorithms. DNA methylation analysis of saliva determines the degree of DNA methylation (the cytosine of the CpG dinucleotide is methylated). The degree of DNA methylation is correlated with aging and is used as another measure of the biological age of the participant.

### Inclusion Criteria:

Men 45-65 years
Female 45-65 years

### Exclusion Criteria:

Females of child-bearing age
Pregnant females
Lactating females
Diabetics
Congestive Heart Disease
Myocardial Infarction in the previous year
Undergoing treatment for cancer
Morbidly Obese
Patient groups in the clinical trial

| | | # of | | Dose (# of |
|---|---|---|---|---|
| Group | Test Article | Volunteers | Gender | Tablets) |
| 1 | Rejuvant ™for Men | 20 | Male | 2 |
| 2 | Placebo for Men | 20 | Male | 2 |
| 3 | Rejuvant ™ for Women | 20 | Female | 2 |
| 4 | Placebo for Women | 20 | Female | 2 |

Primary outcome measures are used to establish the safety of oral administration of the therapeutic composition. Safety and tolerability of single and multiple doses of a composition described herein is measured by adverse events (AEs), physical examinations (PE), vital signs (VS), laboratory safety tests, urinalysis, and 12-lead electrocardiograms (ECG). Outcomes measured during site visits include: height, weight, blood pressure and pulse, complete blood counts, comprehensive metabolic panel, lipid panel, and blood measures such as hemoglobin A1C, c-reactive protein levels, and uric acid.

Complete Blood Count may include: WBC (White Blood Cell Count), Hemoglobin, Hematocrit, Platelet Count/L, MCV (Mean Corpuscular Volume), MCH (Mean Corpuscular Hemoglobin), MCHC (Mean Corpuscular Hemoglobin Concentration), RBC (Red Blood Cell Count), RDW-CV (Red Cell Distribution Width), MPV (Mean Platelet Volume), Neutrophils, Neutrophil %, Lymphocyte Count, Lymphocyte %, Monocyte Count, Monocyte %, Eosinophils, Eosinophil %, Basophils, Basophil %.

Comprehensive Metabolic Panel may include measuring: Sodium, Potassium, Chloride, Carbon Dioxide (CO₂), Urea Nitrogen (BUN), Creatinine, Glucose, Calcium, AST (Aspartate Aminotransferase), ALT (Alanine Aminotransferase), Alk Phos (Alkaline Phosphatase), Bilirubin, Total, Alkaline Phosphatase, Albumin, Protein, Total, Anion Gap, Glomerular Filtration Rate (eGFR), MDRD Estimate.

Lipid Panel may include: Total Cholesterol, LDL (Low Density Lipoprotein) Cholesterol, HDL (High Density Lipoprotein) Cholesterol, Triglyceride,

The secondary outcome are to observe changes in blood chemistries, metabolites, and the amount of DNA methylation by the oral administration of Rejuvant products per dosing described. The secondary outcome measures may include Metabolic Screen (from blood samples), DNA Methylation Assay (from saliva samples).

### Example Questionnaire

How do you feel today?
How would you describe your daily activity?
How would you rate your physical energy level?
How would you rate your cardiovascular endurance level?
How would you describe your ability to focus?
How would you rate your level of memory recall?
How would you describe your quality of sleep?
How would you rate the youthfulness of your hair?
How many minutes per week do you exercise including light exercise?
Is there any other relevant information about the product you'd like to share?

1.-29. (canceled)

30. A method for treating age-related illness, for increasing longevity, or for a combination thereof in a host animal, the method comprising administering a therapeutically effective amount of Ca-AKG and one or more B vitamins to the host animal.

31. A pharmaceutical composition comprising Ca-AKG and one or more B vitamins, and optionally one or more carriers, vehicles, diluents, adjuvants, or excipients, or a combination thereof.

32. A unit dose in single or divided form, the unit dose comprising a therapeutically effective amount of Ca-AKG and one or more B vitamins, and adapted for or configured for use in treating age-related illness, for increasing longevity, or for a combination thereof in a host animal.

33. The method of claim 30 wherein the one or more B vitamins are selected from the group consisting of vitamin B2, B6, B9, and B12, and combinations thereof.

34. The method of claim 30 wherein the one or more B vitamins are selected from the group consisting of vitamin B6 and B12, and combinations thereof.

35. The method of claim 30 wherein the age-related illness is frailty.

36. The method of claim 30 wherein the host animal exhibits a deficiency in blood levels of vitamin B6 or B12, or both B6 and B12.

37. The method of claim 30 wherein the host animal has single polynucleotide polymorphism in MTHFR.

38. The method of claim 30 wherein the single polynucleotide polymorphism in MTHFR is A/A, homozygous positive.

39. The method of claim 30 wherein the single polynucleotide polymorphism in MTHFR is A/G, heterozygous positive.

40. The method of claim 30 wherein the single polynucleotide polymorphism in MTHFR is C667T.

41. The method of claim 30 wherein the single polynucleotide polymorphism in MTHFR is A1298C.

42. The method of claim 30 wherein the host animal has single polynucleotide polymorphism in ALPL.

43. The method of claim 30 wherein the single polynucleotide polymorphism in ALPL is C/C, homozygous positive.

44. The method of claim 30 wherein the single polynucleotide polymorphism in ALPL is C/T, heterozygous positive.

45. The method of claim 30 wherein the host animal has single polynucleotide polymorphism in FUT2.

46. The method of claim 30 wherein the single polynucleotide polymorphism in ALPL is G/G, homozygous positive.

47. The method of claim 30 wherein the single polynucleotide polymorphism in ALPL is A/G, heterozygous positive.

48. The method of claim 30 wherein the therapeutically effective amount is capable of decreasing the biological age of the host animal.

49. The method of claim 30 wherein the therapeutically effective amount is capable of improving the DNA methylation profile of the host animal.
